(19) United States
(12) Patent Application Publication
(10) Pub. No.: US 2019/0302118 A1
(43) Pub. Date: Oct. 3, 2019

(54) LIPID BIOMARKERS FOR THE DIAGNOSIS OF CANCER

(71) Applicants: AGENCY FOR SCIENCE, TECHNOLOGY AND RESEARCH, Singapore (SG); SINGAPORE HEALTH SERVICES PTE LTD, Singapore (SG)

(72) Inventors: Ying Swan HO, Singapore (SG); Lian Yee YIP, Singapore (SE); Kiat Hon Tony LIM, Singapore (SG); Shao Weng Daniel TAN, Singapore (SG); Xulei YANG, Singapore (SG); Si Yong YEO, Singapore (SG)

(21) Appl. No.: 16/083,878

(22) PCT Filed: Mar. 10, 2017
(86) PCT No.: PCT/SG2017/050120

§ 371 Date: Sep. 10, 2018

(30) Foreign Application Priority Data
Mar. 10, 2016 (SG) 10201601873S

(51) Int. Cl. G01N 33/574; G01N 33/92

(52) U.S. Cl. CPC G01N 33/574; G01N 33/92; G01N 2405/08; G01N 2405/02; G01N 2405/04

(57) ABSTRACT

The present disclosure provides a cancer biomarker for a patient suffering from pleural effusion, a method of determining whether a patient suffering from pleural effusion has cancer, and a method of treating cancer in a patient suffering from pleural effusion. Disclosed herein also include a cancer biomarker for the detection of cancer with EGFR mutation, a method of determining whether a patient suffering from cancer has EGFR mutation, and a method of treating cancer in a patient with EGFR mutation.

### CROSS-REFERENCE TO RELATED APPLICATIONS

This patent application is a U.S. National Phase Application under 35 U.S.C. § 371 of International Application No. PCT/SG2017/050120, filed on 10 Mar. 2017, entitled LIPID BIOMARKERS FOR THE DIAGNOSIS OF CANCER, which claims the benefit of priority of Singapore Provisional Application No. 10201601873S, filed on 10 Mar. 2016, the contents of it were incorporated by reference in the entirety for all purposes.

### FIELD OF THE INVENTION

The present invention relates to biochemistry in particular biomarkers. In particular, the present invention relates to biomarkers associated with cancer, particularly lung cancer, breast cancer, gastric cancer and squamous cell carcinoma, and methods of using the biomarkers to determine whether a patient suffering from pleural effusion has cancer, in particular cancer with EGFR mutation.

### BACKGROUND OF THE INVENTION

Cancer is the second leading cause of death worldwide, accounting for 8.2 million deaths in 2012. Cancer mortality can be significantly reduced if detected and treated early. Methods for reliable detection of cancer mainly involve the use of endoscopies, radioactive scannings and tissue biopsy, which are expensive and invasive procedures that impose certain health risks to the patient. Hence there is a need to provide a non-invasive method for effectively detecting cancer in a patient.

A pleural effusion is a build-up of fluid in the pleural space, an area between the layers of tissue that line the lungs and the chest cavity. Pleural effusion can be associated and hence as an indication for various cancers such as lung cancer, breast cancer, gastric cancer and squamous cell carcinoma. However, pleural effusion can also be a manifestation of benign inflammatory conditions including pneumonia, tuberculosis and pulmonary disorders. As such, cytological detection of malignant cells in lung pleural effusion forms a cornerstone in the diagnosis of cancer in a patient suffering from pleural effusion. However, the diagnostic performance of cytology is dependent on the tumor type, tumor burden in the pleural space and the expertise of the cytologist. In pleural effusion samples with low cell yields, diagnostic accuracy can be compromised, resulting in false-negative rates of more than 30%.

Therefore, there is a need to provide biomarkers independent of cell numbers in order to complement and improve the accuracy of cancer diagnosis in patients suffering from pleural effusion.

In addition to the diagnosis of cancers, the ability to stratify cancer cases into subtypes based on specific gene mutations is critical to pharmacological intervention as the appropriate treatment relates to the driver mutation of the cancer. In particular, specific mutations in the epidermal growth factor receptor (EGFR) have been reported to be one of the top driver oncogenes in cancers. Cancers with EGFR mutations have shown increased sensitivity to tyrosine kinase inhibitors (TKIs) such as gefitinib and erlotinib, making such medications a more effective treatment option than standard chemotherapy. At present, EGFR mutations are most commonly detected based on DNA extracts obtained from tumor tissue samples, although DNA extracted from malignant pleural effusion supernatant has been suggested as a potential alternative sample. One key challenge with using malignant pleural effusion for EGFR testing has been the large variation in quantity and quality of the DNA present in such samples, which can result in lower sensitivities in comparison to tissue samples. Consequently, alternative biomarkers in malignant pleural effusion indicative of EGFR mutations are needed for the selection of cases for EGFR mutation targeted cancer treatment.

### SUMMARY OF THE INVENTION

In one aspect, there is provided a cancer biomarker for a patient suffering from pleural effusion, wherein the biomarker is at least two selected from the group consisting of: fatty acid (22:6), hydroxyl fatty acid (16:0), fatty acid (20:1), fatty acid (18:2), fatty acid (18:1), fatty acid (16:1), fatty acid (20:5), fatty acid (22:4), fatty acid (22:5), fatty acid (20:4) and fatty acid (20:2), and wherein the cancer is selected from lung cancer, breast cancer, gastric cancer and squamous cell carcinoma.

In another aspect, there is provided a method of determining whether a patient suffering from pleural effusion has cancer, the method comprising: (i) measuring the concentration of the cancer biomarker of the present invention in a sample obtained from the patient; (ii) comparing the concentration of the cancer biomarker in (i) with the concentration of the same cancer biomarker in a sample obtained from a control group, wherein an increased concentration of the cancer biomarker in (i) as compared to the control group indicates that the patient has cancer, wherein the control group comprises a patient suffering from pleural effusion without cancer, and wherein the cancer is selected from lung cancer, breast cancer, gastric cancer and squamous cell carcinoma.

In another aspect, there is provided a method of treating cancer in a patient suffering from pleural effusion, the method comprising: (i) measuring the concentration of the cancer biomarker of the present invention in a sample obtained from the patient; (ii) comparing the concentration of the cancer biomarker in (i) with the concentration of the same cancer biomarker in a sample obtained from a control group, wherein the control group comprises a patient suffering from pleural effusion without cancer; and (iii) administering to the patient at least one anti-cancer treatment, if there is an increased concentration of the cancer biomarker in (i) as compared to the control group; wherein the cancer is selected from lung cancer, breast cancer, gastric cancer and squamous cell carcinoma.

In another aspect, there is provided a cancer biomarker for the detection of cancer with EGFR mutation, wherein the biomarker is at least two selected from the group consisting of: fatty acid (20:5), fatty acid (22:5), fatty acid (18:1), fatty acid (18:3), phosphatidylcholine (38:8), phosphatidylcholine (40:8), phosphatidylcholine (41:6), phosphatidylethanolamine (P-36:5), phosphatidylcholine (36:5), phosphatidylcholine (P-36:5), fatty acid (22:4), fatty acid (23:0), phosphatidylethanolamine (38:4), triacylglycerol 54:8 and Gb3(42:2), and wherein the cancer is selected from lung cancer, breast cancer, gastric cancer and squamous cell carcinoma.

In another aspect, there is provided a method of determining whether a patient suffering from cancer has EGFR mutation, the method comprising: (i) measuring the concentration of the cancer biomarker of the present invention in a sample obtained from the patient; (ii) comparing the concentration of the cancer biomarker in (i) with the concentration of the same cancer biomarker in a sample obtained from a control group, wherein an increased concentration of the cancer biomarker in (i) as compared to the control group indicates that the patient has EGFR mutation, wherein the control group comprises a patient suffering from cancer without EGFR mutation, and wherein the cancer is selected from lung cancer, breast cancer, gastric cancer and squamous cell carcinoma.

In another aspect, there is provided a method of treating cancer in a patient with EGFR mutation, the method comprising: (i) measuring the concentration of the cancer biomarker of the present invention in a sample obtained from the patient; (ii) comparing the concentration of the cancer biomarker in (i) with the concentration of the same cancer biomarker in a sample obtained from a control group; and (iii) administering to the patient at least one anti-cancer treatment for cancer with EGFR mutation if there is an increased concentration of the cancer biomarker in (i) as compared to the control group; wherein the control group comprises a patient suffering from cancer without EGFR mutation, and wherein the cancer is selected from lung cancer, breast cancer, gastric cancer and squamous cell carcinoma.

### BRIEF DESCRIPTION OF THE DRAWINGS

The invention will be better understood with reference to the detailed description when considered in conjunction with the non-limiting examples and the accompanying drawings, in which:

FIG. 1A shows the principal component analysis (PCA) score plots for benign (n=30) and malignant lung pleural effusion (n=36). Light grey, black and dark grey circles represent the benign pleural effusion, malignant pleural effusion without EGFR mutation, and malignant pleural effusion with EGFR mutation, respectively. The PCA analysis of the pleural effusion lipidomes revealed distinctive clustering of the benign and malignant cases, indicating the existence of metabolic differences between these two groups. FIG. 1B shows the PCA score plots for malignant pleural effusions with EGFR mutation (n=19) and without EGFR mutation (n=17). Black and dark grey circles represent the malignant pleural effusion without EGFR mutation, and malignant pleural effusion with EGFR mutation, respectively. The PCA analysis revealed further compositional differences in the lipidomes between the malignant pleural effusion cases with and without EGFR mutations. FIG. 1C shows a heat map of differential lipid metabolites (grouped according to their lipid classes) derived from individual pairwise comparisons between benign, malignant pleural effusion without EGFR mutation and malignant pleural effusion with EGFR mutation. The results show that all represented species are statistically significant (VIP>1, p-value<0.05, fold change (FC) 1.5) for at least one of the pairwise comparisons. The candidate malignancy markers are predominantly in higher abundance in the pleural effusion samples with EGFR mutations, as compared to those without EGFR mutations.

FIG. 2A shows a receiver operating characteristic (ROC) curve of malignant versus benign subjects for individual lipid markers in the class of fatty acids, particularly unsaturated fatty acids, for example, FA (14:2), FA (18:1), FA (18:2), FA (18:3), FA (20:5), FA (22:4), FA (22:5), FA (22:6) and hydroxyl FA (16:0). The results show that each of these fatty acids can be used to discriminate between malignant and benign pleural effusions, with AUC values ranging from 0.74 to 0.87. FIG. 2B shows an ROC curve of malignant versus benign subjects for individual lipid markers in the class of sphingolipids, for example, GalCer (40:1)/GlcCer (40:1), SM (44:1) and SM (42:2). The results show that each of these sphingolipids can be used to discriminate between malignant and benign pleural effusions, with AUC values ranging from 0.66 to 0.73. FIG. 2C shows an ROC curve of malignant versus benign subjects for an optimal combination of four lipid malignancy markers (FA (22:6), FA (22:5), FA (23:0) and Gb3 (42:2) derived from SVM modelling. The results show that this combination of four lipid markers can be used to discriminate between malignant and benign pleural effusions, with an AUC value of 0.94.

FIG. 3A shows a dot plot of the relative levels of FA (22:6) in benign pleural effusion, malignant pleural effusion without EGFR mutation, and malignant pleural effusion with EGFR mutation. Expression levels of FA(22:6) in benign pleural effusion samples are used as the reference point. p-value is calculated based on Mann-Whitney U test, where *denotes p<0.05, **denotes p<0.01, ***denotes p<0.001. The results demonstrate that polyunsaturated fatty acid FA (22:6) is predominantly in higher abundance in malignant pleural effusion samples with EGFR mutation. FIG. 3B shows a dot plot of the relative levels of FA (20:5) in benign pleural effusion, malignant pleural effusion without EGFR mutation, and malignant pleural effusion with EGFR mutation. Expression levels of FA(20:5) in benign pleural effusion samples are used as the reference point. p-value is calculated based on Mann-Whitney U test, where *denotes p<0.05, **denotes p<0.01, ***denotes p<0.001. The results demonstrate that polyunsaturated fatty acid FA (20:5) is predominantly in higher abundance in malignant pleural effusion samples with EGFR mutation. FIG. 3C shows an ROC curve of malignant pleural effusion without EGFR mutation versus malignant pleural effusion with EGFR mutation, in the class of fatty acids (exemplary fatty acids FA (20:3), FA (20:5), FA (22:5) and FA (22:6)). The results demonstrate that each of the exemplary fatty acids can be used to discriminate between malignant pleural effusion with and without EGFR mutation, with AUC ranging from 0.68 to 0.78 (see Table 14). FIG. 3D shows an ROC curve of malignant pleural effusion without EGFR mutation versus malignant pleural effusion with EGFR mutation, in the class of phospholipids (exemplary phospholipids LysoPEtn (P-16:0), PC (41:6) and PEtn (P-36:5)). The results demonstrate that each of the exemplary phospholipids can be used to discriminate between malignant pleural effusion with and without EGFR mutation, with AUC ranging from 0.67 to 0.70 (see Table 14). FIG. 3E shows an ROC curve of malignant pleural effusion without EGFR mutation versus malignant pleural effusion with EGFR mutation, using a combination of seven lipid markers derived from SVM modelling. The seven lipid markers are: FA (20:5), FA (22:4), FA (22:5), FA (23:0), PC (41:6), PEtn (38:4) and Gb3 (42:2). The results demonstrate that the combination of these seven lipid markers can be used to discriminate between malignant pleural effusion with and without EGFR mutation, with an AUC of 0.86 (with 95% confidence interval of 0.73-1.00).

FIG. 4A shows a dot plot of accuracy and AUC for a subset of panel markers in discriminating malignant pleural effusion from benign pleural effusion (total n=128). The marker combinations are derived from SMV models. Identities of the lipid markers in each subset of panel markers are listed in Table 1. FIG. 4B shows an ROC curve of malignant pleural effusion subjects versus benign pleural effusion subjects for an exemplary combination of four panel markers (FA (22:6), Hydroxyl FA (16:0), FA (20:1) and FA (18:2)) derived from SMV model. The AUC is 0.88 (with 95% confidence interval of 0.82-0.95). FIG. 4C shows the corresponding dot plot of FIG. 4B.

FIG. 5A shows a dot plot of accuracy and AUC for a subset of panel markers in discriminating malignant pleural effusion from benign pleural effusion (total n=149). The marker combinations are derived from SMV models. Identities of the lipid markers in each subset of panel markers are listed in Table 2. FIG. 5B shows an ROC curve of malignant pleural effusion subjects versus benign pleural effusion subjects for an exemplary combination of four panel markers (FA (22:6), Hydroxyl FA (16:0), FA (18:2) and FA (18:1)) derived from SMV model. The AUC is 0.89 (with 95% confidence interval of 0.83-0.95). FIG. 5C shows the corresponding dot plot of FIG. 5B.

### DETAILED DESCRIPTION OF THE PRESENT INVENTION

The inventors of the present disclosure have set out to provide alternative biomarkers for the detection of cancers, in particular lung cancer, breast cancer, gastric cancer and squamous cell carcinoma, in a patient suffering from pleural effusion. Lipidomics was used to identify biomarkers and lipid fingerprints that can be used for the detection of cancers. Accordingly, in one aspect, there is provided a cancer biomarker for a patient suffering from pleural effusion, wherein the biomarker is at least two selected from the group consisting of: fatty acid (22:6), hydroxyl fatty acid (16:0), fatty acid (20:1), fatty acid (18:2), fatty acid (18:1), fatty acid (16:1), fatty acid (20:5), fatty acid (22:4), fatty acid (22:5), fatty acid (20:4) and fatty acid (20:2), and wherein the cancer is selected from lung cancer, breast cancer, gastric cancer and squamous cell carcinoma. In some examples, the cancer biomarker of the first aspect comprises at least one fatty acid, preferably one unsaturated fatty acid, more preferably one polyunsaturated fatty acid. In some examples, the cancer biomarker of the first aspect comprises at least FA (22:6) and one other fatty acid. In some examples, the cancer biomarker of the first aspect comprises at least FA (22:6) and hydroxyl fatty acid (16:0). In some other examples, the cancer biomarker of the first aspect can further comprise fatty acid FA (23:0) and/or FA (18:3). Some exemplary cancer biomarkers of the first aspect are listed in Table 1.

TABLE 1
Exemplary marker combinations capable of discriminating pleural effusion
of malignant subjects from benign subjects (derived from SVM models).

| No. of fatty | | | |
|---|---|---|---|
| acids in | Accuracy | AUC | |
| combination | (%) | (%) | Fatty acid combinations |
| 2 | 82 | 89 | FA 22:6 + FA 16:0 (OH) |
| 3 | 81 | 89 | FA 22:6 + FA 16:0 (OH) + FA 18:2 |
| 4 | 82 | 89 | FA 22:6 + FA 16:0 (OH) + FA 18:2 + |
| | | | FA 18:1 |
| 5 | 80 | 89 | FA 22:6 + FA 16:0 (OH) + FA 18:2 + |
| | | | FA 18:1 + FA 22:5 |
| 6 | 81 | 89 | FA 22:6 + FA 16:0 (OH) + FA 18:2 + |
| | | | FA 18:1 + FA 22:5 + FA 20:4 |
| 7 | 81 | 89 | FA 22:6 + FA 16:0 (OH) + FA 18:2 + |
| | | | FA 18:1 + FA 22:5 + FA 20:4 + FA |
| | | | 20:2 |
| 8 | 80 | 88 | FA 22:6 + FA 16:0 (OH) + FA 18:2 + |
| | | | FA 18:1 + FA 22:5 + FA 20:4 + FA |
| | | | 20:2 + FA 20:1 |
| 9 | 80 | 89 | FA 22.6 + FA 16:0 (OH) + FA 18:2 + |
| | | | FA 18:1 + FA 22:5 + FA 20:4 + FA |
| | | | 20:2 + FA 20:1 + FA 20:5 |
| 10 | 79 | 88 | FA 22.6 + FA 16:0 (OH) + FA 18:2 + |
| | | | FA 18:1 + FA 22:5 + FA 20:4 + FA |
| | | | 20:2 + FA 20:1 + FA 20:5 + FA 22:4 |
| 11 | 80 | 86 | FA 22:6 + FA 16:0 (OH) + FA 18:2 + |
| | | | FA 18:1 + FA 22:5 + FA 20.4 + FA |
| | | | 20.2 + FA 20.1 + FA 20:5 + FA 22:4 + |
| | | | FA 16:1 |

Other than fatty acids, the cancer biomarker of the first aspect can comprise other classes of lipids, including but not limited to ceramides, lysophospholipids, phosphatidylcholines (PCs), phosphatidylethanolamines (PEs) and triacylglycerols (TAGs). Examples of these other classes of lipids are listed in Tables 6 to 9. Exemplary cancer biomarkers containing the combinations of fatty acids and the above other classes of lipids are listed in Table 2.

TABLE 2
Marker combinations capable of discriminating pleural effusion of
malignant subjects from benign subjects (derived from SVM models).

| No. of fatty | | | |
|---|---|---|---|
| acids in | Accuracy | AUC | |
| combination | (%) | (%) | Fatty acid combinations |
| 3 | 77 | 88 | FA22:6 + FA22:5 + FA23:0 |
| 4 | 86 | 94 | FA22:6 + FA22:5 + FA23:0 + Gb3(42:2) |
| 5 | 83 | 94 | FA22:6 + FA22:5 + FA23:0 + |
| | | | Gb3(42:2) + FA18:2 |
| 6 | 83 | 94 | FA22:6 + FA22:5 + FA23:0 + |
| | | | Gb3(42:2) + FA18:2 + HydroxlFA16:0 |
| 7 | 89 | 95 | FA22:6 + FA22:5 + FA23:0 + |
| | | | Gb3(42:2) + FA18:2 + HydroxlFA16:0 + |
| | | | LysoPE(P-18:0) |
| 8 | 89 | 95 | FA22:6 + FA22:5 + FA23:0 + |
| | | | Gb3(42:2) + FA18:2 + HydroxlFA16:0 + |
| | | | LysoPE(P-18:0) + PC(o-36:1) |
| 9 | 83 | 94 | FA22:6 + FA22:5 + FA23:0 + |
| | | | Gb3(42:2) + FA18:2 + HydroxlFA16:0 + |
| | | | LysoPE(P-18:0) + PC(o-36:1) + |
| | | | LysoPC22:6 |
| 10 | 86 | 93 | FA22:6 + FA22:5 + FA23:0 + |
| | | | Gb3(42:2) + FA18:2 + HydroxlFA16:0 + |
| | | | LysoPE(P-18:0) + PC(o-36:1) + |
| | | | LysoPC22:6 + PE(38:4) |
| 11 | 86 | 94 | FA22:6 + FA22:5 + FA23:0 + |
| | | | Gb3(42:2) + FA18:2 + HydroxlFA16:0 + |
| | | | LysoPE(P-18:0) + PC(o-36:1) + |
| | | | LysoPC22:6 + PE(38:4) + FA18:3 |
| 12 | 86 | 93 | FA22:6 + FA22:5 + FA23:0 + |
| | | | Gb3(42:2) + FA18:2 + HydroxlFA16:0 + |
| | | | LysoPE(P-18:0) + PC(o-36:1) + |
| | | | LysoPC22:6 + PE(38:4) + FA18:3 + |
| | | | Gb3(34:1) |
| 13 | 85 | 93 | FA22:6 + FA22:5 + FA23:0 + |
| | | | Gb3(42:2) + FA18:2 + HydroxlFA16:0 + |
| | | | LysoPE(P-18:0) + PC(o-36:1) + |
| | | | LysoPC22:6 + PE(38:4) + FA18:3 + |
| | | | Gb3(34:1) + AcylCar18:2 |
| 14 | 85 | 94 | FA22:6 + FA22:5 + FA23:0 + |
| | | | Gb3(42:2) + FA18:2 + HydroxlFA16:0 + |
| | | | LysoPE(P-18:0) + PC(o-36:1) + |
| | | | LysoPC22:6 + PE(38:4) + FA18:3 + |
| | | | Gb3(34:1) + AcylCar18:2 + SM(42:2) |
| 15 | 85 | 95 | FA22:6 + FA22:5 + FA23:0 + |
| | | | Gb3(42:2) + FA18:2 + HydroxlFA16:0 + |
| | | | LysoPE(P-18:0) + PC(o-36:1) + |
| | | | LysoPC22:6 + PE(38:4) + FA18:3 + |
| | | | Gb3(34:1) + AcylCar18:2 + SM(42:2) + |
| | | | GalCer(40:1)/GlcCer(40:1) |

As used herein, the term “patient” or “subject” or “individual”, which may be used interchangeably, relates to animals, for example mammals, including cows, horses, non-human primates, dogs, cats and humans. The patient of the present disclosure may be suffering from pleural effusion, and may be suspected of suffering or may have previously suffered from cancer, such as lung cancer, breast cancer, gastric cancer and squamous cell carcinoma. For example, the method of the present invention may be applied to a subject with pleural effusion suspected of suffering from lung cancer. In another example, the method of the present disclosure may be applied to a subject suspected of having recurrence of cancer. The term “recurrence” as used herein refers to the return of or redetection of cancer in a patient who has been deemed to be free of cancer.

The term “lung cancer” or “lung carcinoma” as used interchangeably herein, refers to a malignant lung tumor characterized by uncontrolled cell growth in tissues of the lung. If left untreated, this growth can spread beyond the lung by the process of metastasis into nearby tissue or other parts of the body. Most cancers that start in the lung, known as primary lung cancers, are carcinomas. Secondary lung cancers are a result of metastasis of other primary cancers, such as breast cancers, gastric cancers. The two main types are small-cell lung carcinoma (SCLC) and non-small-cell lung carcinoma (NSCLC). SCLC usually presents in the central airways and infiltrates the submucosa leading to narrowing of bronchial airways. Compared NSCLC, SCLC has a shorter doubling time, higher growth fraction, and earlier development of metastases. NSCLC is any type of epithelial lung cancer other than SCLC. Example of NSCLC include squamous cell lung cancer, large cell lung carcinoma, and adenocarcinoma of the lung.

Some specific examples of the cancer biomarker of the first aspect that can be used for the detection of NSCLC are listed in Table 3.

TABLE 3
Exemplary marker combinations capable of discriminating pleural effusion
of NSCLC patients from benign subjects (derived from SVM models).

| No. of fatty | | | |
|---|---|---|---|
| acids in | Accuracy | AUC | |
| combination | (%) | (%) | Fatty acid combinations |
| 2 | 78 | 87 | FA 22:6 + FA 16:0 (OH) |
| 3 | 79 | 88 | FA 22:6 + FA 16:0 (OH) + FA 20:1 |
| 4 | 80 | 88 | FA 22:6 + FA 16:0 (OH) + FA 20:1 + |
| | | | FA 18:2 |
| 5 | 80 | 89 | FA 22:6 + FA 16:0 (OH) + FA 20:1 + |
| | | | FA 18:2 + FA 18:1 |
| 6 | 80 | 88 | FA 22:6 + FA 16:0 (OH) + FA 20:1 + |
| | | | FA 18:2 + FA 18:1 + FA 16:1 |
| 7 | 80 | 88 | FA 22:6 + FA 16:0 (OH) + FA 20:1 + |
| | | | FA 18:2 + FA 18:1 + FA 16:1 + FA |
| | | | 20:5 |
| 8 | 80 | 88 | FA 22:6 + FA 16:0 (OH) + FA 20:1 + |
| | | | FA 18:2 + FA 18:1 + FA 16:1 + FA |
| | | | 20:5 + FA 22:4 |
| 9 | 80 | 87 | FA 22:6 + FA 16:0 (OH) + FA 20:1 + |
| | | | FA 18:2 + FA 18:1 + FA 16:1 + FA |
| | | | 20:5 + FA 22:4 + FA 22:5 |
| 10 | 80 | 87 | FA 22:6 + FA 16:0 (OH) + FA 20:1 + |
| | | | FA 18:2 + FA 18:1 + FA 16:1 + FA |
| | | | 20:5 + FA 22:4 + FA 22:5 + FA 20:4 |
| 11 | 80 | 86 | FA 22:6 + FA 16:0 (OH) + FA 20:1 + |
| | | | FA 18:2 + FA 18:1 + FA 16:1 + FA |
| | | | 20:5 + FA 22:4 + FA 22:5 + FA 20:4 + |
| | | | FA 20:2 |

The term “breast cancer” as used herein refers to the cancer that forms in the breast tissue. Breast cancer usually starts off in the inner lining of milk ducts or the lobules that supply them with milk. A breast cancer that originates in the lobules is known as lobular carcinoma, while one that originates in the ducts is called ductal carcinoma. Breast cancer cells can spread into the lungs by the process of metastasis, resulting in secondary lung cancers.

The term “gastric cancer” as used herein refers to the cancer that originates from the cells lining the inner mucosal layer of the stomach. Gastric cancer cells can spread through the muscular and serosal layers of the stomach before metastasizing to lymph nodes and distant organs such as the liver and lungs, resulting in secondary cancers in these distant organs.

The terms “squamous cell carcinoma” and “squamous cell cancer” as used interchangeably herein refers to the cancer that originates from the squamous cells, which are thin, flat cells that represent the shape of fish scales found in the tissue that forms the surface of the skin, the lining of the hollow organs of the body, and the lining of the respiratory and digestive tracts. One specific example is a squamous cell lung cancer, which is a histological subtype of non-small cell lung cancer.

As used herein, the term “biomarker” refers to molecular indicators of a specific biological property, a biochemical feature or facet that can be used to determine the presence or absence and/or severity of a particular disease or condition. In the present disclosure, the term “biomarker” refers to a group of at least two or at least three lipids, derivatives or metabolites thereof, which are associated with cancer, in particular lung cancer, breast cancer and, gastric cancer and squamous cell carcinoma.

The term “lipid” as used here in refers to a diverse group of naturally occurring organic compounds that are related by their solubility in nonpolar organic solvents (e.g. ether, chloroform, acetone & benzene) and general insolubility in water. The main classes of lipids include fatty acids, ceramides, lysophospholipids, phosphatidylcholines (PCs), phosphatidylethanolamines (PEs) and triacylglycerols (TAGs).

The term “fatty acids” as used herein refers to a diverse group of molecules, usually a carboxylic acid with an aliphatic (or hydrocarbon) chain, formed by chain-elongation of, for example, an acetyl-CoA primer molecule with malonyl coenzyme A (malonyl-CoA) or methylmalonyl-CoA groups in a process called fatty acid synthesis. They are made of a hydrocarbon chain that terminates with a carboxylic acid group. The carbon chain may be saturated or unsaturated, and may be attached to functional groups containing, but not limited to, oxygen, halogens, nitrogen, and sulphur. Fatty acids differ from one another in terms of the length of the hydrocarbon chain, the degree of unsaturation (which is the number of carbon to carbon double bonds present in the hydrocarbon chain), and the position of the double bond(s) within the hydrocarbon chain. In some examples, the number of carbon atoms in the hydrocarbon chain of a fatty acid is between 4 to 26, or 6 to 24, or 8 to 22, or 10 to 20, or 12 to 18, or 14 to 16, or 4, 6, 8, 10, 12, 14, 16, 18, 20, 22, 24, 26, 28, 30, 32, 34, 36, 38, 40, 42, 44, 46, 48, 50, 52, 54, 56, 58, 60, or more. Due to the nature of fatty acid synthesis, the number of carbon atoms in a chain is generally an even number, that is, the number of carbon atoms in an unsaturated fatty acid is a multiple of 2 carbon atoms. The number of double bonds present in the aliphatic chain is between 0, 1, 2, 3, 4, 5, 6, 7, 8, 9, and 10. The determination of the number of the double bonds present in the aliphatic chain of an unsaturated fatty acid is dependent on the valence of the remaining carbon atoms and the actual length of the aliphatic chain. A person skilled in the art would understand that the degree of unsaturation (which is the number of double bonds within an aliphatic chain of a fatty acid) is dependent on the length of the aliphatic chain. For example, an unsaturated fatty acid with 6 carbon atoms in its aliphatic chain can have up to 2 double bonds present in its aliphatic chain as valency permits. Therefore, an aliphatic chain with 6 carbon atoms may have 0 double bonds, 1 double bond or 2 double bonds. Based on the different nomenclatures available for naming fatty acids, the naming may or may not provide information as to the location of the double bond within the aliphatic chain. Three of the commonly used nomenclatures for fatty acids are the lipid number (“C:D”) nomenclature, the delta-x (Δ^{x}) nomenclature and the omega-x (ω-x) nomenclature. For example, in the lipid number nomenclature, “C” represents the number of carbon atoms present in the aliphatic chain, and “D” represents the number of carbon to carbon double bonds (C═C) present. In this representation, the nomenclature does not indicate the exact location of the double bond within the aliphatic chain. For example, an unsaturated fatty acid with the lipid number 18:3 (18 carbon atoms on the chain, with 3 double bonds present) includes both α-linolenic acid and γ-linolenic acid. Using the same example, the delta-x (Δ^{x}) nomenclature for α-linolenic acid is Δ^{9,12,15} and γ-Linolenic acid is Δ^{6,9,12}, with the “x” thereby denoting the position of the double bond as counted from the carboxylic acid end. The delta-x nomenclature may or may not include configuration information of the molecule, for example if the double bond results in a cis- or trans-confirmation of the unsaturated fatty acid. The omega-x nomenclature indicates the location of the double bond by counting from the methyl end (the ω end) of the aliphatic chain. Unless otherwise stated, the nomenclature herein is the lipid number nomenclature.

Fatty acids that could be used as biomarkers of the present disclosure include saturated fatty acids and unsaturated fatty acids. The term “saturated fatty acids” refers to the fatty acids that only have single bonds (i.e. no carbon double bonds, n=0). The term “unsaturated fatty acids” refers to fatty acids that have at least one carbon double bond, i.e. n>1. Examples of fatty acids and derivatives identified in human pleural effusion, their structures and names are listed in Table 4.

TABLE 4
Examples of fatty acids and derivatives of fatty acids identified in human
pleural effusion

| | Examples of fatty acids within the same | Molecular |
|---|---|---|
| Lipid Name | lipid name (common name) | Formula |
| FA(14:2) | 5,8-Tetradecadienoic acid | C₁₄H₂₄O₂ |
| FA(16:1) | Hexadecenoic acid, for example palmitoleic | C₁₆H₃₀O₂ |
| | acid, sapienic acid, 13-Hexadecenoic acid | |
| FA(16:2) | 7Z,10Z-Hexadecadienoic acid | C₁₆H₂₈O₂ |
| FA(18:1) | Oleic acid, vaccenic acid, elaidic acid | C₁₈H₃₄O₂ |
| FA(18:2) | Linoleic acid, Linoelaidic acid | C₁₈H₃₂O₂ |
| FA(18:3) | Linolenic acid, for example, α-linolenic acid, | |
| | γ-linolenic acid, calendic acid | C₁₈H₃₀O₂ |
| FA(20:1) | Eicosenoic acid, for example paullinic acid, | C₂₀H₃₈O₂ |
| | gondoic acid, gadoleic acid | |
| FA(20:2) | Eicosadienoic acid | C₂₀H₃₆O₂ |
| FA(20:3) | Eicosatrienoic acid, 5,8,11-eicosatrienoic acid | C₂₀H₃₄O₂ |
| | (mead acid), dihomo-γ-linolenic acid (DGLA) | |
| FA(20:4) | Eicosatetraenoic acid, for example, arachidonic | C₂₀H₃₂O₂ |
| | acid (all-cis 5,8,11,14-eicosatetraenoic acid), | |
| | all-cis 8,11,14,17-eicosatetraenoic acid | |
| FA(20:5) | Eicosapentaenoic acid (EPA) | C₂₀H₃₀O₂ |
| FA(22:4) | Adrenic acid | C₂₂H₃₆O₂ |
| FA(22:5) | Docosapentaenoic acid (DPA), clupanodonic | C₂₂H₃₂O₂ |
| | acid | |
| FA(22:6) | Docosahexaenoic acid (DHA) | C₂₂H₃₂O₂ |
| FA(23:0) | Tricosanoic acid | C₂₃H₄₆O₂ |
| Hydroxyl | (R)-3-Hydroxy-hexadecanoic acid | C₁₆H₃₂O₃ |
| FA(16:0) | 16-Hydroxy hexadecanoic acid | |
| Trihydroxyl | 9,12,13-Trihydroxy-10-octadecenoic acid, | C₁₈H₃₄O₅ |
| FA(18:1) | 9,10,13-Trihydroxy-11-octadecenoic acid | |

The term “ceramide” as used herein refers to a family of waxy lipid molecules. A ceramide is composed of sphingosine and a fatty acid. Ceramides are found in high concentrations within the cell membrane of cells, since they are component lipids that make up sphingomyelin, one of the major lipids in the lipid bilayer. Ceramide can participate in a variety of cellular signaling: examples include regulating differentiation, proliferation, and programmed cell death (PCD) of cells. Ceramides can be represented by the general formula below, where R represents the alkyl group of a fatty acid:

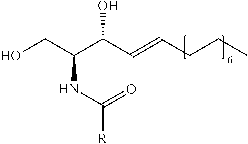

Examples of ceramides identified in human pleural effusion, their structures and names are listed in Table 5.

TABLE 5
Examples of ceramides identified in human pleural effusion

| Lipid Name | Common Name | Molecular Formula |
|---|---|---|
| GalCer(40:1) | Galactosylceramide (d18:1/22:0) | C₄₆H₈₉NO₈ |
| GlcCer(40:1) | Glucosylceramide (d18:1/22:0) | C₄₆H₈₉NO₈ |
| Gb3(34:1) | Trihexosylceramide (d18:1/16:0) | C₅₂H₉₇NO₁₈ |
| Gb3(42:2) | Trihexosylceramide (d18:1/24:1) | C₆₀H₁₁₁NO₁₈ |
| SM(42:2) | Sphingomyelin (42:2) | C₄₇H₉₃N₂O₆P |
| SM(44:1) | Sphingomyelin (44:1) | C₄₉H₁₀₀N₂O₆P⁺ |

The term “galactosylceramides (GalCer)” as used herein refers to non-acidic monoglycosphingolipids, i.e. a sphingolipids with one carbohydrate moiety attached to a ceramide unit. GalCer is generated from ceramide via the enzyme UDP-galactose ceramide galactosyltransferase.

The term “trihexosylceramide” as used herein refers to a glycosphingolipid which contains a trisaccharide (galactose-galactose-glucose) moiety bound in glycosidic linkage to the hydroxyl group of ceramide as the polar head group.

The term “sphingomyelin” as used herein refers to a type of sphingolipid found in animal cell membranes, especially in the membranous myelin sheath that surrounds some nerve cell axons. It usually consists of phosphocholine and ceramide, or a phosphoethanolamine head group; therefore, sphingomyelins can also be classified as sphingophospholipids.

The term “lysophospholipid” as used herein refers to a derivative of a phospholipid in which one or both acyl derivatives have been removed by hydrolysis. Examples of lysophospholipids identified in human pleural effusion, their structures and names are listed in Table 6.

TABLE 6
Examples of lysophospholipids identified in human pleural effusion

| | | Molecular |
|---|---|---|
| Lipid Name | Common Name | Formula |
| LysoPC(P-16:0) | Lysophosphatidylcholine(P-16:0) | C₂₄H₅₀NO₇P |
| LysoPC (22:6) | Lysophosphatidylcholine(22:6) | C₃₀H₅₀NO₇P |
| LysoPE(P-16:0) | Lysophosphatidylethanolamine (P-16:0) | C₂₁H₄₄NO₇P |
| LysoPE(P-18:0) | Lysophosphatidylethanolamine (P-18:0) | C₂₃H₄₈NO₇P |

The term “lysophosphatidylcholine” as used herein refers to derivatives of phosphatidylcholines obtained by their partial hydrolysis that removes one of the fatty acid moieties.

The term “lysophosphatidylethanolamine” as used herein refers to derivatives of phosphatidylethanolamines obtained by their partial hydrolysis that removes one of the fatty acid moieties.

The term “phosphatidylcholine” or “PC” in short form, is used herein to refer to a class of phospholipids that is composed of a choline head group and glycerophosphoric acid, with a variety of fatty acids, including saturated fatty acids and unsaturated fatty acids. The term “choline” as used herein refers to the class of quaternary ammonium salts containing the N,N,N-trimethylethanolammonium cation, represented by the following general formula, where X− on the right denotes an undefined counteranion:

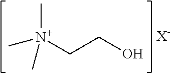

Examples of phosphatidylcholines identified in human pleural effusion, their structures and names are listed in Table 7.

TABLE 7
Examples of phosphatidylcholines identified in human pleural effusion

| Lipid Name | Common Name | Molecular Formula |
|---|---|---|
| PC(34:2) | Phosphatidylcholine (34:2) | C₄₂H₈₀NO₈P |
| PC(36:5) | Phosphatidylcholine (36:5) | C₄₄H₇₈NO₈P |
| PC(38:8) | Phosphatidylcholine (38:8) | C₄₆H₇₆NO₈P |
| PC(41:6) | Phosphatidylcholine (41:6) | C₄₉H₈₆NO₈P |
| PC(O-36:1) | Phosphatidylcholine (O-36:1) | C₄₄H₈₈NO₇P |
| PC(O-44:4) | Phosphatidylcholine (O-44:4) | C₅₂H₉₈NO₇P |
| PC(P-32:1) | Phosphatidylcholine (P-32:1) | C₄₀H₈₀NO₇P |
| PC(P-36:5) | Phosphatidylcholine (P-36:5) | C₄₄H₇₈NO₇P |
| PC(P-38:6) | Phosphatidylcholine (P-38:6) | C₄₆H₈₀NO₇P |
| PC(P-40:7) | Phosphatidylcholine (P-40:7) | C₄₈H₈₂NO₇P |
| PC(40:8) | Phosphatidylcholine (40:8) | C₄₈H₈₀NO₈P |

The term “phosphatidylethanolamines” or “PE” in short form, is used herein to refer to a class of phospholipids consisting of a combination of glycerol esterified with two fatty acids and phosphoric acid. The phosphate group is combined with ethanolamine. The two fatty acids may be the same, or different, and are usually in the 1,2 positions, and sometimes in the 1,3 positions. Examples of phosphatidylehanolamines identified in human pleural effusion, their structures and names are listed in Table 8.

TABLE 8
Examples of phosphatidylethanolamines identified in human pleural effusion

| Lipid Name | Common Name | Molecular Formula |
|---|---|---|
| PE(34:1) | Phosphatidylethanolamine (34:1) | C₃₉H₇₆NO₈P |
| PE(36:4) | Phosphatidylethanolamine (36:4) | C₄₁H₇₄NO₈P |
| PE(38:4) | Phosphatidylethanolamine (38:4) | C₄₃H₇₈NO₈P |
| PE(P-36:5) | Phosphatidylethanolamine (P-36:5) | C₄₁H₇₂NO₇P |
| PE(P-38:5) | Phosphatidylethanolamine (P-38:5) | C₄₃H₇₆NO₇P |

The term “triacylglycerol” or “TAG” or “TG” in short form, is used herein to refer to an ester derived from glycerol and three fatty acids. In some examples, the three fatty acids are different from each other. In some other examples, at least two of the three fatty acids are the same. In some other examples, all three fatty acids are the same. Triacylglycerols can be either saturated or unsaturated. Examples of triacylglycerols identified in human effusion, their structures and names are listed in Table 9.

TABLE 9
Examples of triacylglycerols identified in human pleural effusion.

| Lipid Name | Common Name | Molecular Formula |
|---|---|---|
| TG(53:7) | Triacylglycerol (53:7) | C₅₇H₉₆O₆ |
| TG(54:6) | Triacylglycerol (54:6) | C₅₇H₉₈O₆ |
| TG(54:7) | Triacylglycerol (54:7) | C₅₇H₉₆O₆ |
| TG(54:8) | Triacylglycerol (54:8) | C₅₇H₉₄O₆ |
| TG(56:8) | Triacylglycerol (56:8) | C₅₉H₉₈O₆ |
| TG(56:9) | Triacylglycerol (56:9) | C₅₉H₉₆O₆ |
| TG(58:0) | Triacylglycerol (58:0) | C₆₁H₁₁₈O₆ |

As illustrated in the Experimental Section of the present disclosure, the inventors of the present disclosure found that the biomarker of the first aspect can be a combination of any 2, 3, 4, 5, 6, 7, 8, 9, 10, or 11-15, or 16-20, or 21-25, 26-30 or all 33 of the following lipids: hydroxyl fatty acid (16:0), fatty acid (14:2), fatty acid (18:1), fatty acid (18:2), fatty acid (18:3), fatty acid (20:5), fatty acid (22:4), fatty acid (22:5), fatty acid (22:6), fatty acid (20:3), fatty acid (20:4), fatty acid (16:1), fatty acid (20:1), fatty acid (20:2), fatty acid (23:0), trihydroxyl fatty acid (18:1), Galactosylceramide (40:1)/Glucosylceramide (40:1), sphingomyelin (44:1), sphingomyelin (42:2), phosphatidylcholine (P-36:5), phosphatidylcholine (P-38:6), phosphatidylcholine (P-32:1), lysophosphatidylcholine (P-16:0), phosphatidylcholine (38:8), phosphatidylcholine (41:6), phosphatidylcholine (o-44:4), lysophosphatidylethanolamine (P-18:0), phosphatidylcholine (o-36:1), lysophosphatidylcholine(22:6), phosphatidylethanolamine (38:4), Gb3(34:1), AcylCar18:2 and Gb3(42:2). Various examples of combinations of these lipid biomarkers are exemplified in the Experimental Section below. In one specific example, the biomarker of the first aspect includes the combination of the following four lipids: fatty acid (22:6), fatty acid (22:5), fatty acid (23:0) and Gb3(42:2). The possibility of combining the lipids as a biomarker of the present disclosure is advantageous, as it would ensure detection of the various types of cancers including lung cancer, breast cancer, gastric cancer and squamous cell carcinoma, which are notorious for having significant genetic heterogeneity and complex somatic mutation between individual subjects.

In some examples, the biomarker of the first aspect comprises at least three selected from the group consisting of: hydroxyl fatty acid (16:0), fatty acid (18:1), fatty acid (18:2), fatty acid (20:5), fatty acid (22:4), fatty acid (22:5), fatty acid (22:6), fatty acid (20:4), fatty acid (16:1), fatty acid (20:1) and fatty acid (20:2). As illustrated in the Experimental Section of the present disclosure, in some examples, the biomarker can be a combination of any 2, 3, 4, 5, 6, 7, 8, 9, 10, or all of the above-mentioned lipids. In some examples, the biomarker of the first aspect comprises at least one unsaturated fatty acid, preferably polyunsaturated fatty acid. In some examples, the unsaturated fatty acid or polyunsaturated fatty acid has 20 or 22 carbon chain length.

In one example, the biomarker of the first aspect can be a combination of two lipids, for example: fatty acid (22:6) and hydroxyl fatty acid (16:0). In some other examples, the biomarker can be a combination of three lipids, for example: fatty acid (22:6), hydroxyl fatty acid (16:0) and fatty acid (18:2); fatty acid (22:6), fatty acid (22:5) and fatty acid (23:0); or fatty acid (22:6), hydroxyl fatty acid (16:0) and fatty acid (20:1). In some other examples, the biomarker can be a combination of four lipids, for example: fatty acid (22:6), hydroxyl fatty acid (16:0), fatty acid (18:2) and fatty acid (18:1); fatty acid (22:6), fatty acid (22:5), fatty acid (23:0) and Gb3(42:2); or fatty acid (22:6), hydroxyl fatty acid (16:0), fatty acid (20:1) and fatty acid (18:2). In some other examples, the biomarker can be a combination of five lipids, for example: fatty acid (22:6), hydroxyl fatty acid (16:0), fatty acid (18:2), fatty acid (18:1) and fatty acid (22:5); fatty acid (22:6), fatty acid (22:5), fatty acid (23:0), Gb3(42:2) and fatty acid (18:2); or fatty acid (22:6), hydroxyl fatty acid (16:0), fatty acid (20:1), fatty acid (18:2) and fatty acid (18:1). In some other examples, the biomarker can be a combination of six lipids, for example: fatty acid (22:6), hydroxyl fatty acid (16:0), fatty acid (18:2), fatty acid (18:1), fatty acid (22:5) and fatty acid (20:4); fatty acid (22:6), fatty acid (22:5), fatty acid (23:0), Gb3(42:2), fatty acid (18:2) and hydroxyl fatty acid (16:0); or fatty acid (22:6), hydroxyl fatty acid (16:0), fatty acid (20:1), fatty acid (18:2), fatty acid (18:1) and fatty acid (16:1). In some other examples, the biomarker can be a combination of seven lipids, for example: fatty acid (22:6), hydroxyl fatty acid (16:0), fatty acid (18:2), fatty acid (18:1), fatty acid (22:5), fatty acid (20:4) and fatty acid (20:2); fatty acid (22:6), fatty acid (22:5), fatty acid (23:0), Gb3(42:2), fatty acid (18:2), hydroxyl fatty acid (16:0) and lysophosphatidylethanolamine (P-18:0); or fatty acid (22:6), hydroxyl fatty acid (16:0), fatty acid (20:1), fatty acid (18:2), fatty acid (18:1), fatty acid (16:1) and fatty acid (20:5). In some other examples, the biomarker can be a combination of eight lipids, for example: fatty acid (22:6), hydroxyl fatty acid (16:0), fatty acid (18:2), fatty acid (18:1), fatty acid (22:5), fatty acid (20:4), fatty acid (20:2) and fatty acid (20:1); fatty acid (22:6), fatty acid (22:5), fatty acid (23:0), Gb3(42:2), fatty acid (18:2), hydroxyl fatty acid (16:0), lysophosphatidylethanolamine (P-18:0) and phosphatidylcholine (o-36:1); or fatty acid (22:6), hydroxyl fatty acid (16:0), fatty acid (20:1), fatty acid (18:2), fatty acid (18:1), fatty acid (16:1), fatty acid (20:5) and fatty acid (22:4). In some other examples, the biomarker can be a combination of nine lipids, for example: fatty acid (22:6), hydroxyl fatty acid (16:0), fatty acid (18:2), fatty acid (18:1), fatty acid (22:5), fatty acid (20:4), fatty acid (20:2), fatty acid (20:1) and fatty acid (20:5); fatty acid (22:6), fatty acid (22:5), fatty acid (23:0), Gb3(42:2), fatty acid (18:2), hydroxyl fatty acid (16:0), lysophosphatidylethanolamine (P-18:0), phosphatidylcholine (o-36:1) and lysophosphatidylcholine (22:6); or fatty acid (22:6), hydroxyl fatty acid (16:0), fatty acid (20:1), fatty acid (18:2), fatty acid (18:1), fatty acid (16:1), fatty acid (20:5), fatty acid (22:4) and fatty acid (22:5). In some other examples, the biomarker can be a combination of ten lipids, for example: fatty acid (22:6), hydroxyl fatty acid (16:0), fatty acid (18:2), fatty acid (18:1), fatty acid (22:5), fatty acid (20:4), fatty acid (20:2), fatty acid (20:1), fatty acid (20:5) and fatty acid (22:4); fatty acid (22:6), fatty acid (22:5), fatty acid (23:0), Gb3(42:2), fatty acid (18:2), hydroxyl fatty acid (16:0), lysophosphatidylethanolamine (P-18:0), phosphatidylcholine (o-36:1), lysophosphatidylcholine (22:6) and phosphatidylethanolamine (38:4); or fatty acid (22:6), hydroxyl fatty acid (16:0), fatty acid (20:1), fatty acid (18:2), fatty acid (18:1), fatty acid (16:1), fatty acid (20:5), fatty acid (22:4), fatty acid (22:5) and fatty acid (20:4). In some other examples, the biomarker can be a combination of eleven lipids, for example: fatty acid (22:6), hydroxyl fatty acid (16:0), fatty acid (18:2), fatty acid (18:1), fatty acid (22:5), fatty acid (20:4), fatty acid (20:2), fatty acid (20:1), fatty acid (20:5), fatty acid (22:4) and fatty acid (16:1); fatty acid (22:6), fatty acid (22:5), fatty acid (23:0), Gb3(42:2), fatty acid (18:2), hydroxyl fatty acid (16:0), lysophosphatidylethanolamine (P-18:0), phosphatidylcholine (o-36:1), lysophosphatidylcholine (22:6), phosphatidylethanolamine (38:4) and fatty acid (18:3); or fatty acid (22:6), hydroxyl fatty acid (16:0), fatty acid (20:1), fatty acid (18:2), fatty acid (18:1), fatty acid (16:1), fatty acid (20:5), fatty acid (22:4), fatty acid (22:5), fatty acid (20:4) and fatty acid (20:2). In some other examples, the biomarker can be a combination of twelve lipids, for example: fatty acid (22:6), fatty acid (22:5), fatty acid (23:0), Gb3(42:2), fatty acid (18:2), hydroxyl fatty acid (16:0), lysophosphatidylethanolamine (P-18:0), phosphatidylcholine (o-36:1), lysophosphatidylcholine (22:6), phosphatidylethanolamine (38:4), fatty acid (18:3) and Gb3(34:1). In some other examples, the biomarker can be a combination of thirteen lipids, for example: fatty acid (22:6), fatty acid (22:5), fatty acid (23:0), Gb3(42:2), fatty acid (18:2), hydroxyl fatty acid (16:0), lysophosphatidylethanolamine (P-18:0), phosphatidylcholine (o-36:1), lysophosphatidylcholine (22:6), phosphatidylethanolamine (38:4), fatty acid (18:3), Gb3(34:1) and AcylCar (18:2). In some other examples, the biomarker can be a combination of fourteen lipids, for example: fatty acid (22:6), fatty acid (22:5), fatty acid (23:0), Gb3(42:2), fatty acid (18:2), hydroxyl fatty acid (16:0), lysophosphatidylethanolamine (P-18:0), phosphatidylcholine (o-36:1), lysophosphatidylcholine (22:6), phosphatidylethanolamine (38:4), fatty acid (18:3), Gb3(34:1), AcylCar (18:2) and sphingomyelin (42:2). In some other examples, the biomarker can be a combination of fifteen lipids, for example: fatty acid (22:6), fatty acid (22:5), fatty acid (23:0), Gb3(42:2), fatty acid (18:2), hydroxyl fatty acid (16:0), lysophosphatidylethanolamine (P-18:0), phosphatidylcholine (o-36:1), lysophosphatidylcholine (22:6), phosphatidylethanolamine (38:4), fatty acid (18:3), Gb3(34:1), AcylCar (18:2), sphingomyelin (42:2) and GalCer(40:1)/GlcCer(40:1).

Since the biomarker of the first aspect could be used for the detection of cancer, accordingly, in a second aspect, there is provided a method of determining whether a patient suffering from pleural effusion has cancer, the method comprising (i) measuring the concentration of the cancer biomarker of the first aspect in a sample obtained from the patient; (ii) comparing the concentration of the cancer biomarker in (i) with the concentration of the same cancer biomarker in a sample obtained from a control group; wherein an increased concentration of the cancer biomarker in (i) as compared to the control group indicates that the patient has cancer; wherein the control group comprises a patient suffering from pleural effusion without cancer; and wherein the cancer is selected from lung cancer, breast cancer, gastric cancer and squamous cell carcinoma.

The term “sample” as used herein refers to a biological sample, or a sample that comprises at least some biological materials such as bodily fluids. In some examples, the biological sample is not a tissue sample or not a sample obtained from tissue biopsy. In some examples, the biological sample is a liquid sample. In one specific example, the biological sample is a pleural fluid sample, or a sample containing pleural fluid.

The term “pleural fluid” as used herein refers to a liquid derived from the blood in the capillaries in the lungs. It is found in small quantities between the layers of the pleural membranes that cover the chest cavity and the outside of each lung. It serves as a lubricant for the movement of the lungs during breathing. A variety of conditions and diseases can cause inflammation of the pleurae and/or excessive accumulation of pleural fluid.

A sample of pleural fluid can be collected using a procedure called thoracentesis. The term “thoracentesis” as used herein refers to a procedure in which a cannula, or a hollow needle is inserted into the pleural space between the lungs and the chest wall. This procedure is done to remove excess fluid, i.e. pleural effusion, from the pleural space, thus allowing the patient suffering from pleural effusion to breathe better. Although it is considered as an invasive procedure, since it is done as part of a routine treatment, no additional invasive procedure is carried out for the purpose of collecting the sample for diagnosis, thus it is more advantageous as compared to other sample collection procedures such as tissue biopsy.

The concentration of the biomarker in the sample could be measured using liquid chromatography-mass spectrometry (LC-MS), an analytical chemistry technique that combines the physical separation capabilities of liquid chromatography with the mass analysis capabilities of mass spectrometry.

LC-MS is a powerful technique that has very high sensitivity, making it useful for the separation, general detection and potential identification of chemicals of particular masses in the presence of other chemicals (i.e., in complex mixtures). LC-MS includes targeted LC-MS and untargeted LC-MS. Targeted LC-MS can be performed when standards required for the quantification of the target analytes are commercially available. Alternatively, surrogate analytes can be used to facilitate quantification of the target analytes if their standards are not commercially available. Examples of surrogate analytes include but are not limited to stable isotope-labeled standards of the target analytes, such as palmitoleic acid (U-¹³C16), linoleic acid (U-¹³C18), oleic acid (U-¹³C18), EPA-d5 and DHA (U-¹³C22). When LC-MS is used for the detection and analysis of the biomarker in a sample, only a small amount of sample is required. For example, the volume of the sample required could be as little as 200 μL, or 150 μL, or 100 μL, or 90 μL, or 80 μL, or 70 μL, or 60 μL, or 50 μL, or 40 μL, or 30 μL, or 20 μL, or 10 μL.

In a third aspect, there is provided a method of treating cancer in a patient suffering from pleural effusion, the method comprising: (i) measuring the concentration of the cancer biomarker of the first aspect in a sample obtained from the patient; (ii) comparing the concentration of the cancer biomarker in (i) with the concentration of the same cancer biomarker in a sample obtained from a control group, wherein the control group comprises a patient suffering from pleural effusion without cancer; and (iii) administering to the patient at least one anti-cancer treatment, if there is an increased concentration of the cancer biomarker in (i) as compared to the control group; wherein the cancer is selected from lung cancer, breast cancer, gastric cancer and squamous cell carcinoma.

As used herein, the terms “treatment”, “treat”, “treating” or “amelioration” refers to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder associated with a malignant condition or cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but can also include a cessation or at least slowing of progress or worsening of symptoms that would be expected in absence of treatment. Beneficial or desired clinical results include, but are not limited to, alleviation of one or more symptom(s) of a malignant disease, diminishment of extent of a malignant disease, stabilized (i.e., not worsening) state of a malignant disease, delay or slowing of progression of a malignant disease, amelioration or palliation of the malignant disease state, and remission (whether partial or total), whether detectable or undetectable. The term “treatment” of a disease also includes providing relief from the symptoms or side effects of the disease (including palliative treatment). The term “anti-cancer treatment” should be construed accordingly. Examples of anti-cancer treatment include but are not limited to chemotherapy, radiotherapy, surgical treatment, immunotherapy and a combination thereof. In one specific example, the anti-cancer treatment comprises the use of an anti-cancer drug.

The inventors of the present disclosure envisaged that some of the lipids markers as disclosed herein could be used to detect whether there are certain mutations, such as mutation in the epidermal growth factor receptor (EGFR), in the cancer cells of a patient. Accordingly, in the fourth aspect, there is provided a cancer biomarker for the detection of cancer with EGFR mutation, wherein the biomarker is at least two selected from the group consisting of: fatty acid (20:5), fatty acid (22:5), fatty acid (18:1), fatty acid (18:3), phosphatidylcholine (38:8), phosphatidylcholine (40:8), phosphatidylcholine (41:6), phosphatidylethanolamine (P-36:5), phosphatidylcholine (36:5), phosphatidylcholine (P-36:5), fatty acid (22:4), fatty acid (23:0), phosphatidylethanolamine (38:4), triacylglycerol 54:8 and Gb3(42:2), and wherein the cancer is selected from lung cancer, breast cancer, gastric cancer and squamous cell carcinoma. In some examples, the cancer biomarker of the fourth aspect comprises at least one fatty acid, preferably one unsaturated fatty acid, more preferably one polyunsaturated fatty acid. In some examples, the cancer biomarker of the fourth aspect comprises at least PC (41:6) and FA (22:5). In some other examples, the cancer biomarker of the fourth aspect can further comprise lipids selected from the group consisting of: fatty acid (20:3), fatty acid (20:4), fatty acid (22:6), lysophosphatidylethanolamine (P-16:0), phosphatidylethanolamine (P-38:5), fatty acid (16:2) and phosphatidylcholine (P-32:1). Some exemplary cancer biomarkers of the fourth aspect are listed in Table 10.

TABLE 10
Exemplary marker combinations capable of discriminating malignant
pleural effusion with and without EGFR mutations (derived from SVM
models)

| No. of fatty | | | |
|---|---|---|---|
| acids in | Accuracy | AUC | |
| combination | (%) | (%) | Fatty acid combinations |
| 2 | 72 | 80 | PC(41:6) + FA22:5 |
| 3 | 81 | 81 | PC(41:6) + FA22:5 + FA23:0 |
| 4 | 78 | 84 | PC(41:6) + FA22:5 + FA23:0 + |
| | | | FA22:4 |
| 5 | 75 | 84 | PC(41:6) + FA22:5 + FA23:0 + |
| | | | FA22:4 + PE(38:4) |
| 6 | 81 | 86 | PC(41.6) + FA22:5 + FA23:0 + |
| | | | FA22:4 + PE(38:4) + Gb3(42:2) |
| 7 | 83 | 86 | PC(41:6) + FA22:5 + FA23:0 + |
| | | | FA22:4 + PE(38:4) + Gb3(42:2) + |
| | | | FA20:5 |
| 8 | 83 | 87 | PC(41:6) + FA22:5 + FA23:0 + |
| | | | FA22:4 + PE(38:4) + Gb3(42:2) + |
| | | | FA20:5 + FA18:1 |
| 9 | 81 | 89 | PC(41:6) + FA22:5 + FA23:0 + |
| | | | FA22:4 + PE(38:4) + Gb3(42:2) + |
| | | | FA20:5 + FA18:1 + PC(P-36:5) |
| 10 | 72 | 83 | PC(41:6) + FA22:5 + FA23:0 + |
| | | | FA22:4 + PE(38:4) + Gb3(42:2) + |
| | | | FA20:5 + FA18:1 + PC(P-36:5) + |
| | | | FA18:3 |
| 11 | 75 | 85 | PC(41:6) + FA22:5 + FA23:0 + |
| | | | FA22:4 + PE(38:4) + Gb3(42:2) + |
| | | | FA20:5 + FA18:1 + PC(P-36:5) + |
| | | | FA18:3 + PE(P-36:5) |
| 12 | 81 | 88 | PC(41:6) + FA22:5 + FA23:0 + |
| | | | FA22:4 + PE(38:4) + Gb3(42:2) + |
| | | | FA20:5 + FA18:1 + PC(P-36:5) + |
| | | | FA18:3 + PE(P-36:5) + PC(38:8) |
| 13 | 81 | 87 | PC(41:6) + FA22:5 + FA23:0 + |
| | | | FA22:4 + PE(38:4) + Gb3(42:2) + |
| | | | FA20:5 + FA18:1 + PC(P-36:5) + |
| | | | FA18:3 + PE(P-36:5) + PC(38:8) + |
| | | | PC(40:8) |
| 14 | 81 | 85 | PC(41:6) + FA22:5 + FA23:0 + |
| | | | FA22:4 + PE(38:4) + Gb3(42:2) + |
| | | | FA20:5 + FA18:1 + PC(P-36:5) + |
| | | | FA18:3 + PE(P-36:5) + PC(38:8) + |
| | | | PC(40:8) + TG(54:8) |
| 15 | 81 | 85 | PC(41:6) + FA22:5 + FA23:0 + |
| | | | FA22:4 + PE(38:4) + Gb3(42:2) + |
| | | | FA20:5 + FA18:1 + PC(P-36:5) + |
| | | | FA18:3 + PE(P-36:5) + PC(38:8) + |
| | | | PC(40:8) + TG(54:8) + PC(36:5) |

As shown in Table 10, in one example, the cancer biomarker of the fourth aspect can be a combination of two lipids, for example: phosphatidylcholine (41:6) and fatty acid (22:5). In another example, the cancer biomarker of the fourth aspect can be a combination of three lipids, for example: phosphatidylcholine (41:6), fatty acid (22:5) and fatty acid (23:0). In another example, the cancer biomarker of the fourth aspect can be a combination of four lipids, for example: phosphatidylcholine (41:6), fatty acid (22:5), fatty acid (23:0) and fatty acid (22:4). In another example, the cancer biomarker of the fourth aspect can be a combination of five lipids, for example: phosphatidylcholine (41:6), fatty acid (22:5), fatty acid (23:0), fatty acid (22:4) and phosphatidylethanolamine (38:4). In another example, the cancer biomarker of the fourth aspect can be a combination of six lipids, for example: phosphatidylcholine (41:6), fatty acid (22:5), fatty acid (23:0), fatty acid (22:4), phosphatidylethanolamine (38:4) and Gb3(42:2). In another example, the cancer biomarker of the fourth aspect can be a combination of seven lipids, for example: phosphatidylcholine (41:6), fatty acid (22:5), fatty acid (23:0), fatty acid (22:4), phosphatidylethanolamine (38:4), Gb3(42:2) and fatty acid (20:5). In another example, the cancer biomarker of the fourth aspect can be a combination of eight lipids, for example: phosphatidylcholine (41:6), fatty acid (22:5), fatty acid (23:0), fatty acid (22:4), phosphatidylethanolamine (38:4), Gb3(42:2), fatty acid (20:5) and fatty acid (18:1). In another example, the cancer biomarker of the fourth aspect can be a combination of nine lipids, for example: phosphatidylcholine (41:6), fatty acid (22:5), fatty acid (23:0), fatty acid (22:4), phosphatidylethanolamine (38:4), Gb3(42:2), fatty acid (20:5), fatty acid (18:1) and phosphatidylcholine (P-36:5). In another example, the cancer biomarker of the fourth aspect can be a combination of ten lipids, for example: phosphatidylcholine (41:6), fatty acid (22:5), fatty acid (23:0), fatty acid (22:4), phosphatidylethanolamine (38:4), Gb3(42:2), fatty acid (20:5), fatty acid (18:1), phosphatidylcholine (P-36:5) and fatty acid (18:3). In another example, the cancer biomarker of the fourth aspect can be a combination of eleven lipids, for example: phosphatidylcholine (41:6), fatty acid (22:5), fatty acid (23:0), fatty acid (22:4), phosphatidylethanolamine (38:4), Gb3(42:2), fatty acid (20:5), fatty acid (18:1), phosphatidylcholine (P-36:5), fatty acid (18:3) and phosphatidylethanolamine (P-36:5). In another example, the cancer biomarker of the fourth aspect can be a combination of twelve lipids, for example: phosphatidylcholine (41:6), fatty acid (22:5), fatty acid (23:0), fatty acid (22:4), phosphatidylethanolamine (38:4), Gb3(42:2), fatty acid (20:5), fatty acid (18:1), phosphatidylcholine (P-36:5), fatty acid (18:3), phosphatidylethanolamine (P-36:5) and phosphatidylcholine (38:8). In another example, the cancer biomarker of the fourth aspect can be a combination of thirteen lipids, for example: phosphatidylcholine (41:6), fatty acid (22:5), fatty acid (23:0), fatty acid (22:4), phosphatidylethanolamine (38:4), Gb3(42:2), fatty acid (20:5), fatty acid (18:1), phosphatidylcholine (P-36:5), fatty acid (18:3), phosphatidylethanolamine (P-36:5), phosphatidylcholine (38:8) and phosphatidylcholine (40:8). In another example, the cancer biomarker of the fourth aspect can be a combination of fourteen lipids, for example: phosphatidylcholine (41:6), fatty acid (22:5), fatty acid (23:0), fatty acid (22:4), phosphatidylethanolamine (38:4), Gb3(42:2), fatty acid (20:5), fatty acid (18:1), phosphatidylcholine (P-36:5), fatty acid (18:3), phosphatidylethanolamine (P-36:5), phosphatidylcholine (38:8), phosphatidylcholine (40:8) and triacylglycerol (54:8). In another example, the cancer biomarker of the fourth aspect can be a combination of fifteen lipids, for example: phosphatidylcholine (41:6), fatty acid (22:5), fatty acid (23:0), fatty acid (22:4), phosphatidylethanolamine (38:4), Gb3(42:2), fatty acid (20:5), fatty acid (18:1), phosphatidylcholine (P-36:5), fatty acid (18:3), phosphatidylethanolamine (P-36:5), phosphatidylcholine (38:8), phosphatidylcholine (40:8), triacylglycerol (54:8) and phosphatidylcholine (36:5).

The term “epidermal growth factor receptor” or “EGFR” in short, is the cell-surface receptor for members of the epidermal growth factor family (EGF family) of extracellular protein ligands. It is a member of the ErbB family of receptors, a subfamily of four closely related receptor tyrosine kinases: EGFR (ErbB-1), HER2/c-neu (ErbB-2), Her 3 (ErbB-3) and Her 4 (ErbB-4). Mutations that lead to EGFR overexpression or overactivity have been associated with a number of cancers. In particular, specific mutations in EGFR have been reported to be one of the top driver oncogenes in NSCLC, with a prevalence of 9-23%. NSCLC cases with EGFR mutations have shown increased sensitivity to tyrosine kinase inhibitors (TKIs) such as gefitinib, erlotinib, afatinib and osimertinib, making such medications a more effective treatment option than standard chemotherapy. At present, EGFR mutations are most commonly detected based on DNA extracts obtained from tumor tissue samples. One key challenge with using pleural effusion for EGFR DNA testing has been the large variation in quantity and quality of the DNA present in the pleural effusion samples, which can result in lower sensitivities in comparison to tissue samples.

As illustrated in the Experimental Section of the present disclosure, the inventors of the present disclosure found that the biomarker of the fourth aspect can be a combination of any 2, 3, 4, 5, 6, 7, 8, 9, 10, 11, 12, 13, 14 or 15 of the following lipids: fatty acid (20:5), fatty acid (22:5), fatty acid (18:1), fatty acid (18:3), phosphatidylcholine (38:8), phosphatidylcholine (40:8), phosphatidylcholine (41:6), phosphatidylethanolamine (P-36:5), phosphatidylcholine (36:5), phosphatidylcholine (P-36:5), fatty acid (22:4), fatty acid (23:0), phosphatidylethanolamine (38:4), triacylglycerol 54:8 and (Gb3(42:2). Various examples of combinations of these lipid biomarkers are exemplified in the Experimental Section below. In some examples, the biomarker of the fourth aspect comprises at least one unsaturated fatty acid, preferably polyunsaturated fatty acid. In some examples, the unsaturated fatty acid or polyunsaturated fatty acid has 20 or 22 carbon chain length. In one example, the biomarker of the fourth aspect includes the combination of the following 7 lipids: fatty acid (20:5), fatty acid (22:4), fatty acid (22:5), fatty acid (23:0), phosphatidylcholine (41:6), PE (38:4) and Gb3 (42:2).

Since the biomarker of the fourth aspect could be used for the detection of EGFR mutations in cancer, accordingly, in a fifth aspect, there is provided a method of determining whether a patient suffering from cancer has EGFR mutation, the method comprising: (i) measuring the concentration of the cancer biomarker of the fourth aspect in a sample obtained from the patient; (ii) comparing the concentration of the cancer biomarker in (i) with the concentration of the same cancer biomarker in a sample obtained from a control group; wherein an increased concentration of the cancer biomarker in (i) as compared to the control group indicates that the patient has EGFR mutation; wherein the control group comprises a patient suffering from cancer without EGFR mutation, and wherein the cancer is selected from lung cancer, breast cancer, gastric cancer and squamous cell carcinoma.

In the sixth aspect, there is provided a method of treating cancer in a patient with EGFR mutation, the method comprising: (i) measuring the concentration of the cancer biomarker of the fourth aspect in a sample obtained from the patient; (ii) comparing the concentration of the cancer biomarker in (i) with the concentration of the same cancer biomarker in a sample obtained from a control group; and (iii) administering to the patient at least one anti-cancer treatment for cancer with EGFR mutation if there is an increased concentration of the cancer biomarker in (i) as compared to the control group; wherein the control group comprises a patient suffering from cancer without EGFR mutation, and wherein the cancer is selected from lung cancer, breast cancer, gastric cancer and squamous cell carcinoma.

Examples of anti-cancer treatment for cancer with EGFR mutation include but are not limited to chemotherapy, radiotherapy, surgical treatment, immunotherapy and combination thereof. In one specific example, the anti-cancer treatment is chemotherapy. In another specific example, the chemotherapy includes the use of a tyrosine kinase inhibitor, such as afatinib, erlotinib, osimertinib and gefitinib.

A person skilled in the art should be able to appreciate that when a patient suffering from cancer has been identified to have EGFR, more than one anti-cancer treatment may be used to treat the patient, with at least one anti-cancer treatment for cancer with EGFR mutation. These treatments could be given to the patient either simultaneously or sequentially. When the different anti-cancer treatments or drugs are administered sequentially, they can be administered immediately after each other, with a time difference in between, or in different treatment cycles. The time difference between each anti-cancer treatment can be 1 hour, or 2 hours, or 3 hours, or 4 hours, or 5 hours, or 6 hours, or 7 hours, or 8 hours, or 9 hours, or 10 hours, or 11 hours, or 12 hours, or 15 hours, or 18 hours, or 21 hours, or 24 hours, or 1 day, or 2 days, or 3 days, or 4 days, or 5 days, or 6 days, or 7 days, or 8 days, or 9 days, or 10 days. When the different anti-cancer treatments are used in different treatment cycles, the time difference between them could be 1 treatment cycle, or 2 treatment cycles, or 3 treatment cycles, or 4 treatment cycles, or 5 treatment cycles, or 6 treatment cycles, or 7 treatment cycles, or 8 treatment cycles, or 9 treatment cycles, or 10 treatment cycles, or 11 treatment cycles, or 12 treatment cycles. In some examples, the second anti-cancer treatment is only administered after the treatment of the patient with the first anti-cancer treatment is completed.

As illustrated in the Experimental section of the present application, the biomarkers of the present application provides a sensitivity and/or specificity of greater than 75%, area under the receiver operating characteristic curve (AUC) of greater than 80%, and overall accuracy of greater than 75%, which are higher than the currently available biomarkers.

As used herein, the singular form “a”, “an” and “the” include plural references unless the context clearly dictates otherwise. For example, the term “a primer” includes a plurality of primers, including mixtures thereof.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain examples may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL EXAMPLES

The following examples illustrate methods by which aspects of the invention may be practiced or materials suitable for practice of certain embodiments of the invention may be prepared.

### Example 1—Lipidomic Profiling of Lung Pleural Effusion Samples

Study Patient and Sample Collection

Lung pleural effusion samples were obtained from 71 patients admitted to Singapore General Hospital and National Cancer Centre Singapore between December 2012 and December 2014. These pleural effusion samples collected via thoracentesis were centrifuged at 805 g at 4° C. for 10 minutes upon collection. The non-small-cell lung cancer (NSCLC) malignant cases were assessed based on clinical diagnosis, cytology of the cells isolated from pleural effusion and histology of tumour tissues. Patients whose histological examinations did not show any malignancy were classified as benign subjects. The patient demographics and characteristics, including age, gender, cytology and histology are provided in Table 11. In our study, there were 30 benign and 41 malignant cases. Of the malignant cases, 19 cases were confirmed to harbour EGFR mutations (EGFR mutant), 17 cases did not possess EGFR mutations (non-EGFR mutant), while the molecular status of the remaining 5 malignant cases were unknown due to insufficient samples for mutation testing following cytology and histology examination. Information on specific EGFR mutations detected for the 19 cases with EGFR mutations are shown in Table 12. All the pleural effusion supernatants were stored at −80° C. prior to LC-MS-based lipidomic analysis. All patients recruited gave informed consent and this study was approved by and carried out in accordance with the guidelines of the Singapore Health Services centralized institutional review board (CIRB 2010/516/B). No statistically significant differences were identified in terms of age (p-value=0.09), gender (p-value=0.15), smoking status (p-value=0.46) and ethnicity (p-value=0.25) between the benign and malignant patients.

TABLE 11
Clinical characteristics of benign subjects (n = 30) and malignant
non-small cell lung cancer patients (n = 41).

| | All | | | | | |
|---|---|---|---|---|---|---|
| | Patients | | Benign | | Malignant | |
| Characteristic | (n = 71) | | (n = 30) | | (n = 41) | |
| Gender | | | | | | |
| Male, % | 35 | (49.3) | 18 | (60.0) | 17 | (41.5) |
| Female, % | 36 | (50.7) | 12 | (40.0) | 24 | (58.5) |
| Age at sample collection, years | | | | | | |
| Mean ± standard deviation | 67 ± 14 | | 63 ± 16 | | 69 ± 12 | |
| Smoking status | | | | | | |
| Smoker (Current/Ex) | 25 | | 9 | | 16 | |
| Non smoker | 46 | | 21 | | 25 | |
| Ethnic group | | | | | | |
| Chinese, % | 61 | (86.0) | 25 | (83.3) | 36 | (87.8) |
| Malay, % | 5 | (7.0) | 1 | (3.3) | 4 | (9.8) |
| Indian, % | 4 | (5.6) | 3 | (10.0) | 1 | (2.4) |
| Others, % | 1 | (1.4) | 1 | (3.3) | 0 | (0.0) |

| Histology | | |
|---|---|---|
| Non-small cell lung adenocarcinoma | 39 | (54.9) |
| Squamous-cell carcinoma | 1 | (1.4) |
| Lymphoepithelioma-like lung | 1 | (1.4) |
| carcinoma | | |
| Non-malignant | 30 | (42.3) |
| Cytology assessment | | |
| Positive for malignancy | 32 | (45.0) |
| Negative for malignancy | 30 | (42.3) |
| Suspicious/atypical confirmed as | 9 | (12.7) |

| malignant based on histology | | | |
|---|---|---|---|
| Mutation subtypes for malignant cases | | | |
| EGFR+ | | 19 | (46.3) |
| EGFR− | N.A. | 17 | (41.5) |
| Unknown | | 5 | (12.2) |

| Subtypes for non-malignant cases | | | |
|---|---|---|---|
| Pneumonia | 22 | (73.3) | |
| Cardiopulmonary congestion | 5 | (16.7) | N.A. |
| Tuberculosis | 3 | (10.0) | |

n, Number of cases; EGFR, Epidermal growth factor receptor

TABLE 12
Information on specific EGFR mutations detected in the 19 NSCLC
patients with EGFR mutations

| Patient | EGFR mutation | |
|---|---|---|
| 1 | Exon 19 deletion | c.2237_2255 |
| 2 | Exon 19 deletion | c.2236_2250 |
| 3 | Exon 19 deletion | c.2239_2250 |
| 4 | Exon 19 deletion | c.2240_2257 |
| 5 | Exon 19 deletion | c.2239_2248 |
| 6 | Exon 19 deletion | c.2236_2250 |
| 7 | Exon 19 deletion | c.2235_2249 |
| 8 | Exon 19 deletion | c.2235_2249 |
| 9 | Exon 19 deletion | c.2235_2249 |
| 10 | Exon 19 deletion | c.2240_2254 |
| 11 | Exon 19 mutation | Not specified |
| | Exon 20 mutation | |
| 12 | Exon 20 insertion | c.2311_2312insTCC |
| 13 | Exon 21 mutation | L858R (c.2573 T > G) |
| 14 | Exon 21 mutation | L858R (c.2573 T > G) |
| 15 | Exon 21 mutation | L858R (c.2573 T > G) |
| 16 | Exon 21 mutation | L858R (c.2573 T > G) |
| 17 | Exon 21 mutation | L858R (c.2573 T > G) |
| 18 | Exon 18 mutation | c.2155 G > A |
| | Exon 21 mutation | L858R (c.2573 T > G) |
| 19 | Not specified | c.2311_2312 |

Reagents and Materials

Reagents were obtained as follows: Optima grade methanol, isopropanol: Fisher Scientific (Loughborough, UK); tricine: Sigma-Aldrich (St Louis, Mo.); ammonia solution “AnalaR” 25%: VWR (Poole, UK); acetonitrile, chloroform, acetic acid, formic acid, ¹³C-labelled isotopic fatty acid standards (palmitoleic acid-¹³C₁₆, palmitic acid-1,2,3,4²³C₄, linoleic acid-¹³C₁₈, stearic acid-¹³C₁₈): Merck (Whitehouse Station, N.J.); odd-chain lipid standards: phosphatidylcholine, PC(9:0/9:0); PC(17:0/17:0); PC(21:0/21:0); phosphatidylethanolamine, PEtn(15:0/15:0); PEtn(17:0/17:0): Avanti Polar Lipids (Alabaster, Ala.). The ¹³C-labelled isotopic and odd-chain lipid standards constitute the lipid reference standard mixture.

Lipid Extraction

50 μL of each pleural effusion sample was aliquoted and 10 μL of lipid reference standard mixture was added to the sample as retention time reference prior to a two-phase modified Bligh and Dyer lipid extraction protocol. Each sample was extracted by sequential addition of methanol, chloroform and 3.8 mM tricine (1:1:0.5 v/v/v, total 2 mL), with sample vortexed for 1 min following each addition. The samples were then centrifuged at 12,000 g at 4° C. for 20 min, following which each sample separated into two fractions—the top methanolic layer contained the polar metabolites while the bottom chloroform layer contained the lipid species. The bottom chloroform fraction enriched with lipid species was collected and stored at −80° C. prior to analysis. Quality control (QC) samples were prepared by mixing equal amount of all the pleural effusion samples and each QC sample was extracted as described above. All samples were randomized for extraction.

Lipidomic Profiling Using LC-MS

An ultra-high performance liquid chromatography (UHPLC) system (Ultimate 3000, ThermoFisher Scientific, MA) interfaced with a Q-Exactive mass spectrometer (ThermoFisher Scientific, MA) was utilized for the lipidomic analysis. Prior to each analytical batch, instrument maintenance (source cleaning and mass calibration) was performed. A reversed phase LC column (Acquity CSH, 1.0×50 mm, 1.7 μm particle size, Waters Corp) was used for separation with two solvents: ‘A’ comprising of acetonitrile, methanol and water (2:2:1) with 0.1% acetic acid and 0.1% ammonia solution, and ‘B’ comprising of isopropanol with 0.1% acetic acid and 0.1% ammonia solution. All samples were dried using a sample concentrator (Bio-techne, Minneapolis, Minn.), reconstituted in a 50:50 (v/v) mixture of solvents A and B. The UHPLC autosampler temperature was set at 4° C. and the injection volume for each sample was 2 μL. All samples were processed in technical triplicates on the LC-MS system. The LC program is as follows: the column was first equilibrated for 1 min at 1% B with a flow rate of 0.1 ml per min. The gradient was increased from 1% B to 82.5% B over 9 min before B was increased to 99% for a 5 min wash at a flow rate of 0.15 ml per min. The column was re-equilibrated for 2.2 min at 1% B. Column temperature was maintained at 45° C. The eluent from the LC system was directed into the mass spectrometer (MS). Electrospray ionization (ESI) in the MS was conducted in both positive and negative modes in full scan with a mass range of 120 to 1800 m/z, resolution of 70,000, automatic gain control (AGC) target of 1×10⁶ ions (ESI+) or 3×10⁶ ions (ESI−), maximum injection time (IT) of 100 ms (ESI+) or 200 ms (ESI−). The heated electrospray ionization (HESI) source used, a spray voltage of 3.75 kV (ESI+) and 3.25 kV (ESI−), capillary temperature of 350° C. (ESI+ and ESI−), sheath gas flow of 25 (ESI+ and ESI−) and auxiliary gas flow of 10 arbitrary units (ESI+ and ESI−).

QC samples were analyzed at regular intervals throughout each batch analysis to monitor the reproducibility of the LC-MS. The extracted samples were re-randomized for LC-MS analysis such that the injection order was independent from the order of sample preparation to minimize systematic bias.

Data Pre-Processing and Statistical Analysis

The raw LC-MS data obtained was then pre-processed and analysed using the XCMS peak finding algorithm. The spiked lipid reference standards had relative standard deviations of less than 20% across all samples, demonstrating the high reproducibility of our extraction and LC-MS method. The QC mixture was used for signal correction between and within each batch analysis. Mass peaks with poor repeatability within the QC samples (coefficient of variation more than 30%) were removed. Total area normalisation (based on ratio of area of each mass peak against sum of peak areas within each sample) was applied to the remaining features in the dataset to correct for minor variations in sample preparation and analysis. The normalised data were exported to SIMCA-P+ (version 13.0.3, Umetrics, Umea, Sweden) for multivariate data analysis to identify potential PE biomarkers. Data were mean-centered and Pareto scaled in SIMCA-P+. Subsequently, an unsupervised principal component analysis (PCA) was utilized to determine the quality of LC-MS data obtained based on the tight clustering of the QC samples and to derive an overview of similarities and differences between individual pleural effusion samples. This was followed by supervised orthogonal projection to latent structures-discriminant analysis (OPLS-DA) where a model was built to identify individual lipid components that were distinctly different between (1) benign, (2) EGFR mutant and (3) non-EGFG mutant malignant cases in a pair-wise manner. These lipid components are identified based on their variable importance for projection (VIP) values. Lipid species with higher VIP (VIP>1) made a greater contribution towards distinguishing the comparator groups in the OPLS-DA model and were considered as potential biomarkers. Univariate analysis was performed using the Mann-Whitney U test at p-value<0.05 to verify the statistical significance of these potential biomarkers. Fold change was calculated by taking the ratio of the peak area contributed by the lipid species of the two comparator groups. These statistical analyses were conducted using Stata/MP 14.0 statistical package (Stata Corp, LP).

Support Vector Machines Modeling

The support vector machines (SVMs) model, is a machine learning technique for pattern recognition. The SVMs construct a boundary that maximizes the distance between the designated class of each sample (e.g. whether the sample is “benign” or “malignant”). An optimal boundary separating the sample class is then defined. In this study, we used the popular libSVM package with linear kernel function to perform the classification, where involved parameters are automatically selected by Bayesian Optimization. The recursive feature elimination (RFE) method, based on backward sequential selection strategy, was used to select the best features of the SVM classifier. Starting with a full candidate set of malignancy lipid markers, features (lipid markers) were removed sequentially such that the variation of separating boundary was minimized and until the desired number of features was reached. Different desired number of features was evaluated to determine the performance of the various feature combinations.

In the construction of a real pattern classification system, the data available are generally limited, such that there is a need for a validation technique to estimate how a classification system will perform in practice. In the present work, the k-fold cross-validation is used to estimate the classification performance. In a single round of k-fold cross-validation, the dataset is first randomly portioned into k subsets (folds), which are of approximately equal size and are mutually exclusive. A SVM classifier is then trained and tested k times, and at each time, one of the subsets is set aside as the testing data while the remaining k-1 subsets set as the training data. In the present, leave-one-out cross validation (that is, k=71) was used.

ROC analyses were then performed for the two optimal combinations of lipid markers capable of differentiating the PE between (i) the benign and malignant patients and (ii) non-EGFR and EGFR mutants. ROC analyses were also performed for the identified lipid species (VIP>1, p-value<0.05, fold change ≥1.5). The ROC is plotted using Stata/MP 14.0 statistical package (Stata Corp, LP) based on the predicted real value of each sample from the trained SVM model.

Metabolite Identification

Mass peaks were first putatively identified based on mass comparison (less than 5 ppm error) with entries from the Kyoto Encyclopedia of Genes and Genome (www.genome.jp/kegg) and the Human Metabolome Database (www.hmdb.ca). Subsequently, the identities of lipid species of interest were verified by MS² spectral comparison with commercially available standards where possible, or by comparison to mass spectral databases available online.

Pleural Effusion Lipidomes

From the lipidomic analysis of benign and malignant pleural effusion samples, a diverse range of lipid species are detected in the human pleural effusion (Tables 4 to 9). These species were identified based on authentic standards or comparison of the characteristic MS² spectra of the respective lipid classes with the online mass spectral databases. The list of identified lipids includes long chain fatty acids, ceramides, phospholipids and triacylglycerols.

Lipidomic Analysis Highlights Key Differences in Benign and Malignant Pleural Effusion

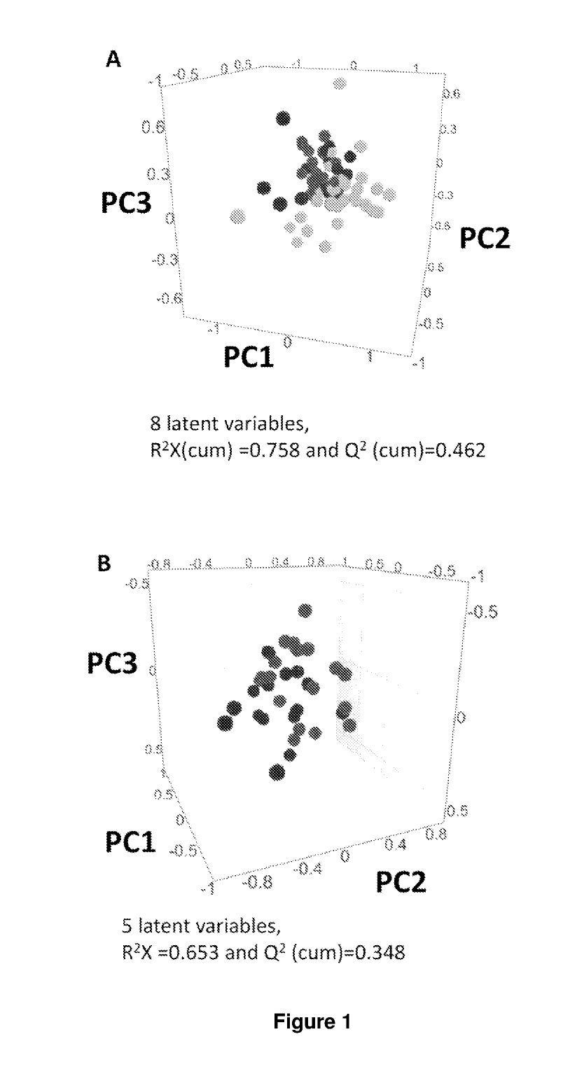

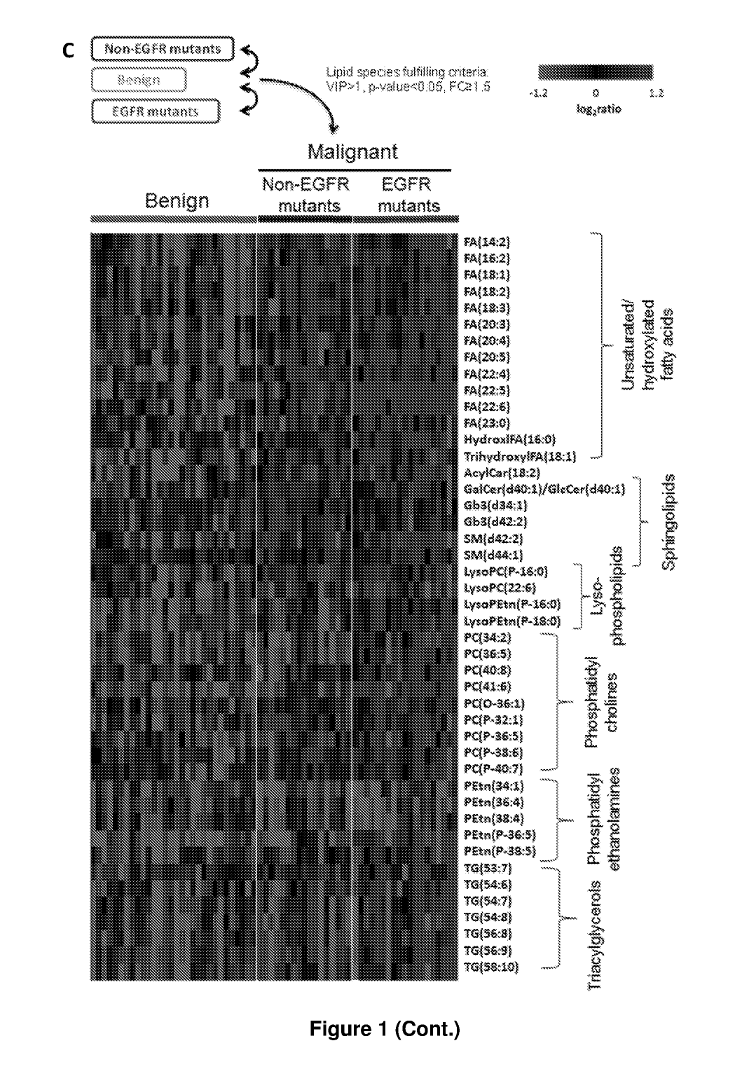

To compare the composition of the lipidomes between the benign and malignant patients, PCA analysis was performed to identify any intrinsic clustering pattern of the pleural effusion samples. The PCA analysis of the pleural effusion lipidomes revealed distinctive clustering of the benign and malignant cases, indicating the existence of metabolic differences between these two groups (FIG. 1A). Interestingly, closer scrutiny of the PCA scores plot for the malignant pleural effusion revealed further compositional differences in the lipidomes between the EGFR mutant and non-EGFR mutant groups within the malignant class (FIG. 1B).

In view of the heterogeneity in the malignant lipidomes between non-EGFR and EGFR mutants, to ensure that reliable indicators of malignancy can be selected, two separate pairwise supervised multivariate analyses were carried out using OPLS-DA subsequently to build models to identify potential lipid differentiators with VIP>1 between: (i) benign vs EGFR mutant and (ii) benign vs non-EGFR mutant. Only lipid species which satisfied the statistical selection criteria for both sets of pair-wise comparisons were shortlisted as candidate markers for malignancy. Additionally, each pairwise multivariate analysis was supplemented by the use of univariate statistical tools (Mann-Whitney U test, fold change analysis). From these analysis, 46 lipid species satisfying the following criteria were identified: VIP>1, p-value<0.05 and an average fold change ≥1.5, in at least one of the pair-wise comparison analysis (FIG. 1C).

Unsaturated fatty acids, phospholipid and sphingolipid constitute some of the major lipid classes discriminating between the malignant from benign pleural effusion samples (FIG. 1C). Within the benign patients, no clear differentiation was observed in the abundance of these lipid markers between the benign infective (pneumonia and tuberculosis) and benign non-infective (cardiopulmonary congestion) pleural effusion samples for markers indicated in the heat map. These lipid species, however, were significantly elevated in the malignant pleural effusion of NSCLC patients compared with the benign pleural effusion cases. Consistent with the earlier observation of a heterogeneous malignant lipidomic profile, the heat map analysis further illustrated the metabolic difference in the malignant lung pleural effusion associated with their genotypes (presence/absence of EGFR mutation) (FIG. 1C). For instance, ether-linked phospholipids such as PC(P-36:5) and PE(P-38:5) were found to be statistically different between benign and EGFR mutant pleural effusion samples but not between benign and non-EGFR mutant cases. Conversely, glycosylated ceramide species including Gb3(42:2) and Gb3(34:1) were found to be significantly elevated in non-EGFR mutant cases, but not in cases with EGFR mutation.

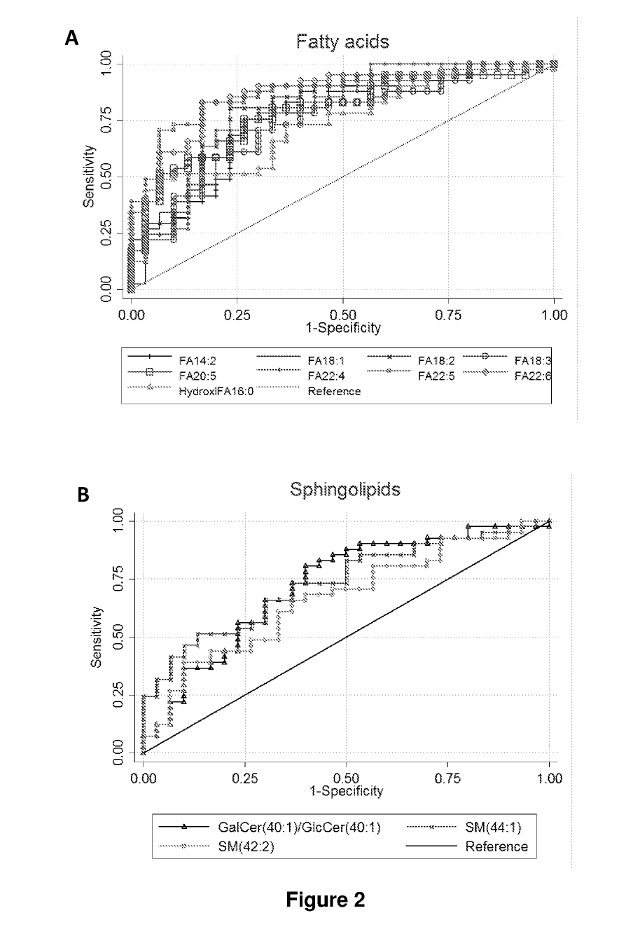

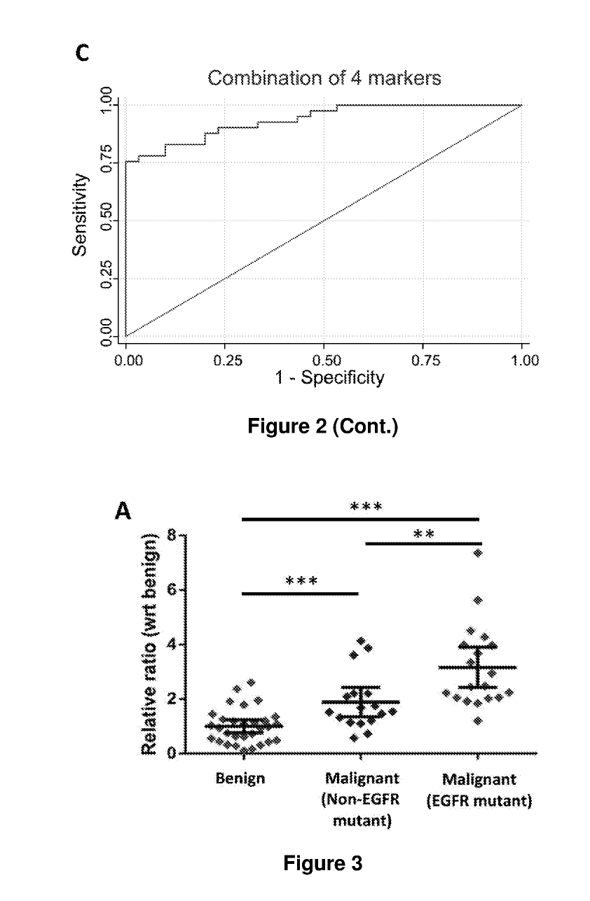

To account for the unique metabotype dependent on the driver mutation for NSCLC, only lipid species satisfying both the abovementioned criteria for “benign vs EGFR mutant” and “benign vs non-EGFR mutants” comparisons were shortlisted as candidate malignancy markers (Table 13). The shortlisted candidates include a large number of unsaturated fatty acids, in addition to specific ceramide and sphingomyelin species. Subsequently, the diagnostic performance of this panel of 12 malignancy lipid species was assessed using ROC curve analysis (FIGS. 2A and 2B, Table 13). Each of these lipid malignancy markers was able to discriminate between the malignant and benign groups with AUC values ranging from 0.66-0.87, sensitivity (SN) of 63.3-82.9%, specificity (SP) of 60.0-83.3% and accuracy (ACC) of 64.8-83.1%. Individual ROC analysis performed on each candidate indicated that the polyunsaturated fatty acids (e.g. FA(22:5), FA(22:6)) gave the best performance as malignancy markers. Combining a number of four malignancy markers comprising of FA(22:6), FA(22:5), FA(23:0) and Gb3(42:2) into a single panel gave a comparable performance than when using the biomarkers alone (AUC=0.94; SN=82.9%; SP=90.0%; ACC=85.9%) (FIG. 2C).

TABLE 13
Summary of potential lipid candidates for malignancy based on separate pair-wise comparisons
of EGFR (n = 19) and non-EGFR mutant (n = 17) cases with benign PE (n = 30).

| | Benign vs | Benign vs | Diagnostic performance as | | | |
|---|---|---|---|---|---|---|
| | non-EGFR | EGFR | malignancy marker | | | |
| | mutants | mutants | | SN | SP | ACC |
| Lipid name^{a} | Ratio^{b} | Ratio^{b} | AUC^{c} | (%) | (%) | (%) |
| Unsaturated/hydroxylated fatty acids | | | | | | |
| Hydroxyl FA(16:0) | 1.56 | 1.83 | 0.74 (0.62-0.85) | 73.17 | 63.33 | 69.01 |
| [3-Hydroxyhexadecanoic acid] | | | | | | |
| FA(14:2) [5,8-Tetradecadienoic acid] | 1.64 | 2.54 | 0.77 (0.65-0.88) | 78.05 | 70.00 | 74.65 |
| FA(18:1) [Oleic acid] | 1.80 | 1.71 | 0.76 (0.65-0.88) | 70.73 | 73.33 | 71.83 |
| FA(18:2) [Linoleic acid] | 1.65 | 1.95 | 0.81 (0.70-0.91) | 80.49 | 76.67 | 78.87 |
| FA(18:3) [Linolenic acid] | 1.53 | 1.81 | 0.74 (0.63-0.86) | 70.73 | 70.00 | 70.42 |
| FA(20:5) [Eicosapentaenoic acid] | 1.82 | 4.43 | 0.79 (0.68-0.90) | 75.61 | 73.33 | 74.65 |
| FA(22:4) [Adrenic acid] | 2.20 | 2.33 | 0.80 (0.69-0.90) | 75.61 | 73.33 | 74.65 |
| FA(22:5) [Docosapentaenoic acid] | 2.46 | 6.11 | 0.87 (0.79-0.96) | 82.93 | 83.33 | 83.10 |
| FA(22:6) [Docosahexaenoic acid] | 1.89 | 3.17 | 0.87 (0.79-0.95) | 82.93 | 83.33 | 83.10 |
| Sphingolipids | 1 | | | | | |
| GalCer(d40:1)/GlcCer(d40:1) | 1.82 | 1.51 | 0.72 (0.60-0.85) | 80.49 | 60.00 | 71.83 |
| SM(d44:1) | 1.53 | 1.53 | 0.73 (0.62-0.85) | 63.33 | 69.01 | 64.79 |
| SM(d42:2) | 1.64 | 1.74 | 0.66 (0.54-0.79) | 63.33 | 64.79 | 69.01 |

^{a}Individual abbreviated lipid names are provided based on the following convention: Lipid class (total number of carbons: total number of double bonds)
^{b}Ratio calculated relative to benign
^{c}AUC value obtained based on receiver operating characteristic (ROC) analysis with 95% confidence interval range provided in parentheses.
VIP, variable importance for projection value;
AUC, area under curve for ROC analysis;
SN, sensitivity;
SP, specificity;
ACC, accuracy;
FA, fatty acid;
GalCer/GlcCer, galactosylceramide/glucosylceramide;
SM, sphingomyelin.

EGFR Mutants Exhibit Greater Metabolic Derangement Compared to Non-EGFR Mutants

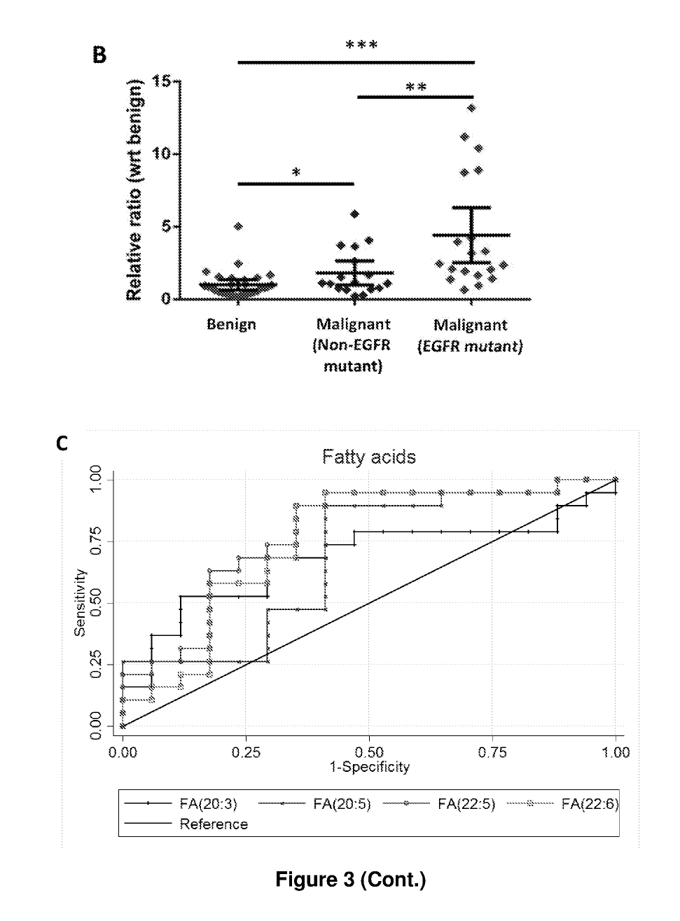

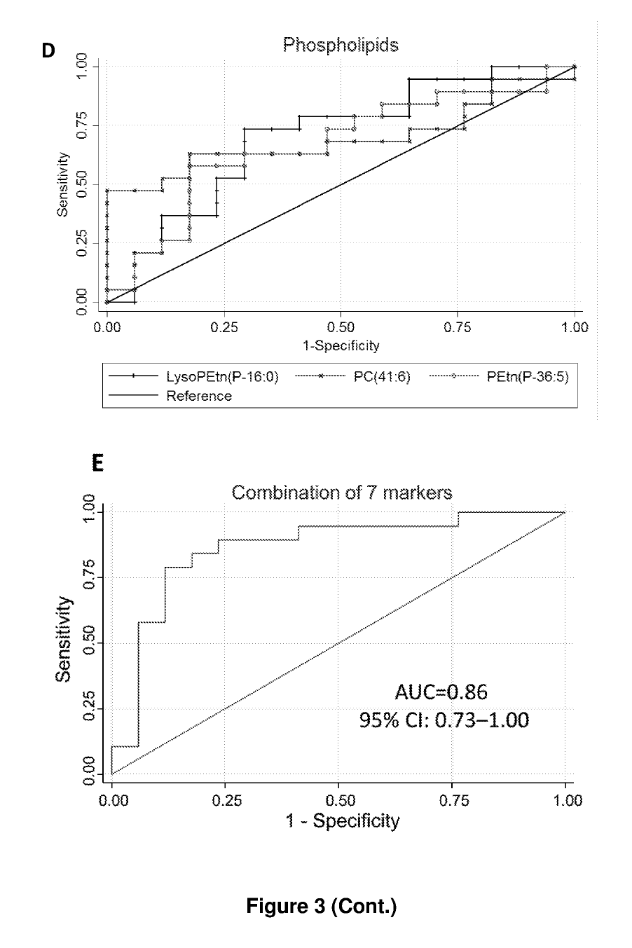

From the earlier analyses, it is noteworthy that almost all the shortlisted malignancy markers were predominantly in higher abundance in the cases with EGFR mutations as compared to those cases without the EGFR mutations (FIG. 1C). The most prominent observations are the increase in levels of polyunsaturated fatty acids comprising of 20 and 22 carbons, as well as ether-linked PEtn and its corresponding lysoPEtn species in cases with EGFR mutations. These trends are exemplified in FIG. 3A to B for FA(22:6) and FA(20:5), illustrating that the pleural effusion samples from patient with EGFR mutations exhibit greater derangement in lipid metabolism compared to those without EGFR mutations. To further understand the influence of NSCLC driver mutations (EGFR/non-EGFR) on the lipidomic profile, a combination of Mann-Whitney U test, OPLS-DA analysis, ROC analyses was performed to identify lipid species that discriminate the cancer cases with EGFR mutations from those without EGFR mutatations (Table 14). Interestingly, the inventors observed that polyunsaturated fatty acids comprising of 20 and 22 carbons and PEtn species were significantly elevated in cases with EGFR mutation by 1.6 to 2.6-fold compared to cases without EGFR mutations (Table 14). Each of these lipid species was able to discriminate between the cases with and without EGFR mutations with AUC ranging from 0.67-0.78, SN of 63.2-89.5%, SP of 58.8-82.4% and ACC of 66.7-75.0% (Table 14, FIGS. 3C and 3D). A combination of 7 lipid species comprising of FA(20:5), FA(22:4), FA(22:5), FA(23:0), PC(41:6), PE(38:4), Gb3(42.2), gave a more superior performance compared to using the biomarkers alone (AUC=0.86; SN=84.2%; SP=82.4%; ACC=83.3%) (FIG. 3E).

TABLE 14
Summary of potential lipid candidates indicative of presence of EGFR
mutation based on separate pair-wise comparisons of EGFR mutant
cases with benign PE and non-EGFR mutant cases.

| | Non-EGFR vs EGFR mutants | | | | |
|---|---|---|---|---|---|
| | Ratio | | | | |
| | (relative to | | SN | SP | ACC |
| Lipid name^{a} | non-EGFR) | AUC^{b} | (%) | (%) | (%) |
| Polyunsaturated | | | | | |
| fatty acids | | | | | |
| FA(20:3) | 1.67 | 0.68 (0.50-0.87) | 68.42 | 70.59 | 69.44 |
| [Eicosatrienoic | | | | | |
| acid] | | | | | |
| FA(20:5) | 2.43 | 0.68 (0.50-0.87) | 89.47 | 58.82 | 75.00 |
| [Eicosapentaenoic | | | | | |
| acid] | | | | | |
| FA(22:5) | 2.49 | 0.78 (0.62-0.94) | 73.68 | 70.59 | 72.22 |
| [Docosapentaenoic | | | | | |
| acid] | | | | | |
| FA(22:6) | 1.68 | 0.75 (0.58-0.93) | 89.47 | 64.71 | 77.78 |
| [Docosahexaenoic | | | | | |
| acid] | | | | | |
| Phospholipids | | | | | |
| LysoPEtn(P-16:0) | 1.57 | 0.70 (0.52-0.88) | 73.68 | 70.59 | 72.22 |
| PC(41:6) | 2.60 | 0.70 (0.51-0.88) | 63.16 | 82.35 | 72.22 |
| PEtn(P-36:5) | 2.30 | 0.67 (0.48-0.85) | 63.16 | 70.59 | 66.67 |

^{a}Individual abbreviated lipid names are provided based on the following convention: Lipid class (total number of carbons: total number of double bonds)
^{b}AUC value obtained based on receiver operating characteristic (ROC) analysis with 95% confidence interval range provided in parentheses.
VIP, variable importance for projection value;
AUC, area under curve for ROC analysis;
SN, sensitivity;
SP, specificity;
ACC, accuracy;
FA, fatty acid;
LysoPEtn(P-), ether-linked lysophosphatidylethanolamine;
PC, phosphatidylcholine;
PEtn(P-), ether-linked phosphatidylethanolamine.

### Example 2—Targeted LC-MS Assay

Study Patient and Sample Collection

Clinical demographics of benign subjects (n=50), malignant NSCLC patients (n=92) and malignant patients of other cancers (n=21) are shown in Table 15. Same sample collection method was used as Example 1.

TABLE 15
Clinical demographics of benign subjects (n = 50), malignant NSCLC
patients (n = 92) and malignant patients of other cancers (n = 21)

| | | | | | Malignant | |
|---|---|---|---|---|---|---|
| | | | Malignant | | (other | |
| | Benign | | NSCLC | | cancers) | |
| Characteristic | (n = 50) | | (n = 92) | | (n = 21) | |
| Gender | | | | | | |
| Male, % | 33 | (66.0) | 45 | (48.9) | 4 | (19.0) |
| Female, % | 17 | (34.0) | 46 | (50.0) | 17 | (81.0) |
| Not specified | NA | | 1 | (1.1) | NA | |
| Age at sample collection, years | | | | | | |
| Mean ± standard deviation | 63 ± 15 | | 66 ± 12 | | 56 ± 9 | |
| Not specified | 2 | | 8 | | 8 | |
| Ethnic group | | | | | | |
| Chinese, % | 35 | (70.0) | 73 | (79.3) | 14 | (66.7) |
| Malay,% | 6 | (12.0) | 7 | (7.6) | 2 | (9.5) |
| Indian, % | 3 | (6.0) | 2 | (2.2) | 1 | (4.8) |
| Others, % | 1 | (2.0) | 5 | (5.4) | 4 | (19.0) |

| | | | | | |
|---|---|---|---|---|---|
| Not specified, % | 5 | (10.0) | 5 | (5.4) | NA |

| | | | |
|---|---|---|---|
| Histology | NA | | |
| Non-small cell lung | | 92 | |
| adenocarcinoma | | | |
| Female cancer* | | | 13 |
| Gastric cancer | | | 2 |
| Squamous cell carcinoma | | | 3 |
| Sarcomatoid neoplasm | | | 1 |
| T cell lymphoma | | | 1 |
| Adenoid cystic carcinoma | | | 1 |

NA: not applicable
*includes breast cancer, fallopian tube cancer, ovarian papillary serous carcinoma, uterus cancer, ovary cancer

Targeted LC-MS-MS analysis was performed using an ultra-high performance liquid chromatography (UHPLC) system (Acquity, Waters Corp) interfaced to a triple quadrupole mass spectrometer (Xevo TQ-S, Waters Corp).

50 μl of sample was first extracted using a liquid-liquid extraction method and the lipid extracts were stored at −80° C. prior to UHPLC-MS analysis. Sample extracts were subsequently dried and reconstituted in a 50:50 mixture of solvents ‘A’ and ‘B’ as described below.

Chromatographic separations were performed using reversed phase Acquity CSH column (1.0×50 mm, 1.7 μm particle size, Waters Corp) with two solvents: ‘A’ comprising of acetonitrile, methanol and water (2:2:1) with 0.1% acetic acid and 0.1% ammonia solution, and ‘B’ comprising of isopropanol with 0.1% acetic acid and 0.1% ammonia solution. All samples were dried using sample concentrator (Bio-techne, Minneapolis, Minn.) and reconstituted in a 50:50 (v/v) mixture of solvents A and B.

Multiple reaction monitoring (MRM) and single ion monitoring (SIM) experiments were performed in ESI negative mode using elution gradient as described in Table 10. Column temperature was maintained at 45° C. and the eluent from the LC system was directed into the MS. The UHPLC autosampler temperature was set at 4° C. and the injection volume for each sample was 2 μL. All samples were processed in technical triplicates on the LC-MS system. The source temperature and desolvation temperature were set at 150° C. and 500° C. respectively. The cone gas flow was 150 L/h and desolvation gas flow was 800 L/h. The capillary voltage was 1.00 kV. The compound-dependent MS parameters for the analytes are shown in Tables 16a and 16b. The chromatographic peak integration was performed using TargetLynx software (Waters Corp).

TABLE 16
Elution condition for LC-MS-MS analysis

| | Time | Solvent A | Solvent | Flow rate |
|---|---|---|---|---|
| Description | (min) | (%) | B (%) | (mL/min) |
| Sample analysis | Initial | 99.0 | 1.0 | 0.1 |
| | 10.0 | 17.5 | 82.5 | 0.1 |
| Column wash | 10.01 | 0.15 | 1.0 | 99.0 |
| | 15.00 | 0.15 | 1.0 | 99.0 |
| | 15.01 | 0.15 | 99.0 | 1.0 |
| | 16.70 | 0.15 | 99.0 | 1.0 |
| Column equilibration | 16.71 | 0.1 | 99.0 | 1.0 |
| | 17.2 | 0.1 | 99.0 | 1.0 |

TABLE 17a
Optimised compound-dependent MS parameters using
Xevo TQ-S MS (SIM)

| | Precursor ion | Dwell |
|---|---|---|
| Metabolite | mass (m/z) | time (s) |
| Hydroxy palmitic acid, FA(16:0)OH | 271 | 0.025 |
| Eicosenoic acid, FA(20:1) | 309 | 0.025 |
| Eicosadienoic acid, FA(20:2) | 307 | 0.025 |

TABLE 17b
Optimised compound-dependent MS parameters using Xevo TQ-S
MS (MRM)

| | | | | | |
|---|---|---|---|---|---|
| | Precursor | Fragment | Dwell | Cone | Collision |
| | ion mass | ion mass | time | voltage | Energy |
| Metabolite | (m/z) | (m/z) | (s) | (V) | (V) |

Analytes

| | | | | | |
|---|---|---|---|---|---|
| Palmitoleic acid, | 253 | 97 | 0.05 | 25 | 20 |
| FA(16:1) | | | | | |
| Oleic acid, | 281 | 263 | 0.025 | 10 | 20 |
| FA(18:1) | | | | | |
| Linoleic acid, | 279 | 59 | 0.025 | 20 | 20 |
| FA(18:2) | | | | | |
| Eicosatetraenoic | 303 | 259 | 0.025 | 50 | 16 |
| acid, FA(20:4) | | | | | |
| Eicosapentaenoic | 301 | 59 | 0.025 | 25 | 20 |
| acid, FA(20:5) | | | | | |
| Adrenic acid, | 331 | 288 | 0.05 | 25 | 20 |
| FA(20:4) | | | | | |
| Docosapentaenoic | 329 | 163 | 0.05 | 25 | 20 |
| acid, FA(22:5) | | | | | |
| Docosahexaenoic | 327 | 59 | 0.05 | 20 | 20 |
| acid, FA(22:6) | | | | | |

Isotopic standards

| | | | | | |
|---|---|---|---|---|---|
| U-¹³C16 | 269 | 103 | 0.05 | 5 | 20 |
| Palmitoleic acid | | | | | |
| U-¹³C18 Linoleic | 297 | 88 | 0.025 | 10 | 20 |
| acid | | | | | |
| U-¹³C18 Oleic acid | 299 | 103 | 0.05 | 30 | 26 |
| Eicosapentaenoic | 306 | 262 | 0.05 | 25 | 10 |
| acid-d5 | | | | | |
| U-¹³C22 | 349 | 173 | 0.05 | 25 | 20 |
| Docosahexaenoic | | | | | |
| acid | | | | | |

Internal standard

| | | | | | |
|---|---|---|---|---|---|
| Lyso | 566 | 267 | 0.025 | 25 | 30 |
| phosphotidylcholine | | | | | |
| 17:1 | | | | | |

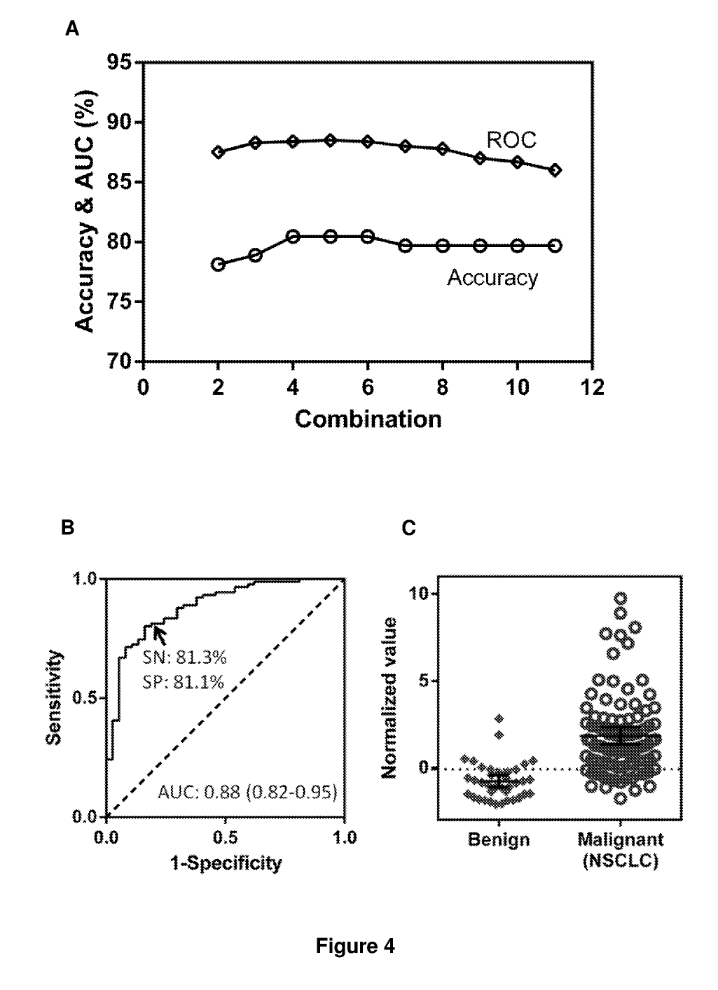

Subsequently, the compound concentrations are fed into multivariate models, such as SVM and PLS-DA, where through leave-one-out cross-validation, it was determined that a combination of 3 or more of these compounds can be used to discriminate between “Benign” and “Malignant” non-small cell lung cancer pleural samples with an average AUC of greater than 0.85, SN and SP of 80% and above (FIG. 4).

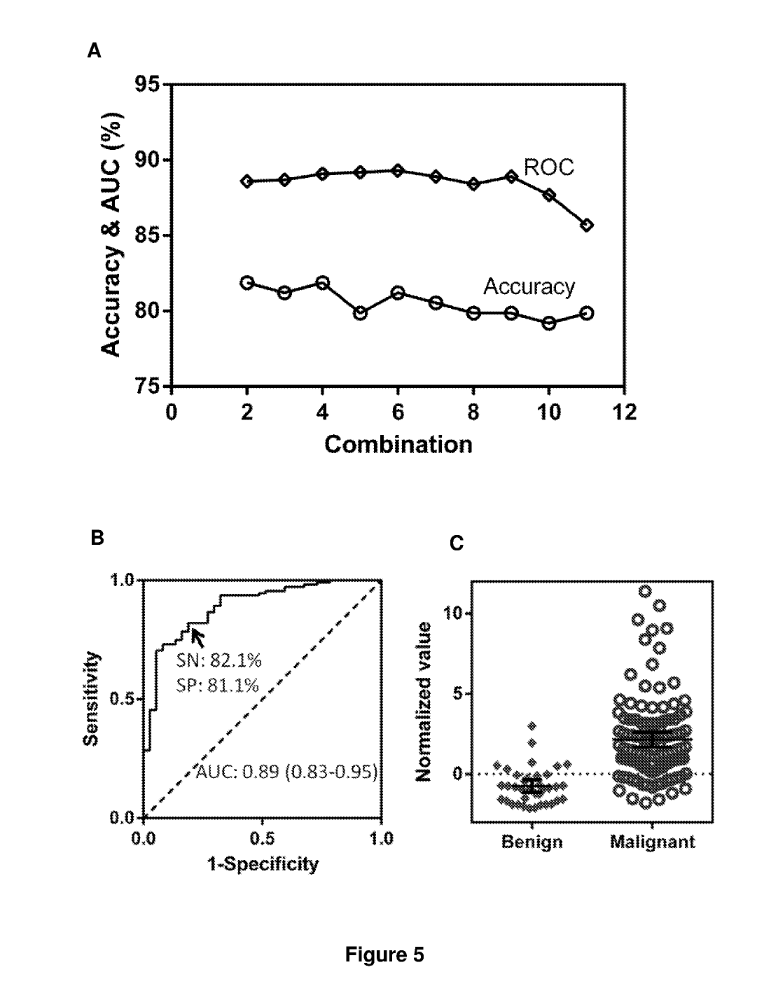

In addition, it was also determined that the combination of 3 or more compounds in Tables 16, 17a and 17b above can also be used to distinguish between “Benign” pleural effusion samples and malignant pleural effusion samples attributed to NSCLC and other cancers (including squamous cell carcinoma, breast and gastric cancers), with an AUC of greater than 0.80, SN and SP of 80% and above (FIG. 5).

1. A cancer biomarker for a patient suffering from pleural effusion, wherein the biomarker is at least two selected from the group consisting of: fatty acid (22:6), hydroxyl fatty acid (16:0), fatty acid (20:1), fatty acid (18:2), fatty acid (18:1), fatty acid (16:1), fatty acid (20:5), fatty acid (22:4), fatty acid (22:5), fatty acid (20:4) and fatty acid (20:2), and wherein the cancer is selected from lung cancer, breast cancer, gastric cancer and squamous cell carcinoma.

2. The cancer biomarker of claim 1, wherein the biomarker is selected from the group consisting of: (1) fatty acid (22:6) and hydroxyl fatty acid (16:0); (2) fatty acid (22:6), hydroxyl fatty acid (16:0) and fatty acid (18:2); (3) fatty acid (22:6), hydroxyl fatty acid (16:0), fatty acid (18:2) and fatty acid (18:1); (4) fatty acid (22:6), hydroxyl fatty acid (16:0), fatty acid (18:2), fatty acid (18:1) and fatty acid (22:5); (5) fatty acid (22:6), hydroxyl fatty acid (16:0), fatty acid (18:2), fatty acid (18:1), fatty acid (22:5) and fatty acid (20:4); (6) fatty acid (22:6), hydroxyl fatty acid (16:0), fatty acid (18:2), fatty acid (18:1), fatty acid (22:5), fatty acid (20:4) and fatty acid (20:2); (7) fatty acid (22:6), hydroxyl fatty acid (16:0), fatty acid (18:2), fatty acid (18:1), fatty acid (22:5), fatty acid (20:4), fatty acid (20:2) and fatty acid (20:1); (8) fatty acid (22:6), hydroxyl fatty acid (16:0), fatty acid (18:2), fatty acid (18:1), fatty acid (22:5), fatty acid (20:4), fatty acid (20:2), fatty acid (20:1) and fatty acid (20:5); (9) fatty acid (22:6), hydroxyl fatty acid (16:0), fatty acid (18:2), fatty acid (18:1), fatty acid (22:5), fatty acid (20:4), fatty acid (20:2), fatty acid (20:1), fatty acid (20:5) and fatty acid (22:4); (10) fatty acid (22:6), hydroxyl fatty acid (16:0), fatty acid (18:2), fatty acid (18:1), fatty acid (22:5), fatty acid (20:4), fatty acid (20:2), fatty acid (20:1), fatty acid (20:5), fatty acid (22:4) and fatty acid (16:1); (11) fatty acid (22:6), hydroxyl fatty acid (16:0) and fatty acid (20:1); (12) fatty acid (22:6), hydroxyl fatty acid (16:0), fatty acid (20:1) and fatty acid (18:2); (13) fatty acid (22:6), hydroxyl fatty acid (16:0), fatty acid (20:1), fatty acid (18:2) and fatty acid (18:1); (14) fatty acid (22:6), hydroxyl fatty acid (16:0), fatty acid (20:1), fatty acid (18:2), fatty acid (18:1) and fatty acid (16:1); (15) fatty acid (22:6), hydroxyl fatty acid (16:0), fatty acid (20:1), fatty acid (18:2), fatty acid (18:1), fatty acid (16:1) and fatty acid (20:5); (16) fatty acid (22:6), hydroxyl fatty acid (16:0), fatty acid (20:1), fatty acid (18:2), fatty acid (18:1), fatty acid (16:1), fatty acid (20:5) and fatty acid (22:4); (17) fatty acid (22:6), hydroxyl fatty acid (16:0), fatty acid (20:1), fatty acid (18:2), fatty acid (18:1), fatty acid (16:1), fatty acid (20:5), fatty acid (22:4) and fatty acid (22:5); and (18) fatty acid (22:6), hydroxyl fatty acid (16:0), fatty acid (20:1), fatty acid (18:2), fatty acid (18:1), fatty acid (16:1), fatty acid (20:5), fatty acid (22:4), fatty acid (22:5) and fatty acid (20:4).

3. The cancer biomarker of claim 1, further comprising FA (23:0) and/or FA (18:3).

4. The cancer biomarker of claim 1, further comprising at least one selected from the group consisting of: Gb3 (42:2), lysophosphatidylethanolamine (P-18:0), phosphatidylcholine (o-36:1), lysophosphatidylcholine (22:6), phosphatidylethanolamine (38:4), Gb3(34:1), AcylCar18:2, sphingomyelin (42:2) and GalCer(40:1)/GlcCer(40:1).

5. The cancer biomarker of claim 4, wherein the biomarker is selected from the group consisting of: (1) fatty acid (22:6), fatty acid (22:5) and fatty acid (23:0); (2) fatty acid (22:6), fatty acid (22:5), fatty acid (23:0) and Gb3(42:2); (3) fatty acid (22:6), fatty acid (22:5), fatty acid (23:0), Gb3(42:2) and fatty acid (18:2); (4) fatty acid (22:6), fatty acid (22:5), fatty acid (23:0), Gb3(42:2), fatty acid (18:2) and hydroxyl fatty acid (16:0); (5) fatty acid (22:6), fatty acid (22:5), fatty acid (23:0), Gb3(42:2), fatty acid (18:2), hydroxyl fatty acid (16:0) and lysophosphatidylethanolamine (P-18:0); (6) fatty acid (22:6), fatty acid (22:5), fatty acid (23:0), Gb3(42:2), fatty acid (18:2), hydroxyl fatty acid (16:0), lysophosphatidylethanolamine (P-18:0) and phosphatidylcholine (0-36:1); (7) fatty acid (22:6), fatty acid (22:5), fatty acid (23:0), Gb3(42:2), fatty acid (18:2), hydroxyl fatty acid (16:0), lysophosphatidylethanolamine (P-18:0), phosphatidylcholine (o-36:1) and lysophosphatidylcholine (22:6); (8) fatty acid (22:6), fatty acid (22:5), fatty acid (23:0), Gb3(42:2), fatty acid (18:2), hydroxyl fatty acid (16:0), lysophosphatidylethanolamine (P-18:0), phosphatidylcholine (o-36:1), lysophosphatidylcholine (22:6) and phosphatidylethanolamine (38:4); (9) fatty acid (22:6), fatty acid (22:5), fatty acid (23:0), Gb3(42:2), fatty acid (18:2), hydroxyl fatty acid (16:0), lysophosphatidylethanolamine (P-18:0), phosphatidylcholine (o-36:1), lysophosphatidylcholine (22:6), phosphatidylethanolamine (38:4) and fatty acid (18:3); (10) fatty acid (22:6), fatty acid (22:5), fatty acid (23:0), Gb3(42:2), fatty acid (18:2), hydroxyl fatty acid (16:0), lysophosphatidylethanolamine (P-18:0), phosphatidylcholine (o-36:1), lysophosphatidylcholine (22:6), phosphatidylethanolamine (38:4), fatty acid (18:3) and Gb3(34:1); (11) fatty acid (22:6), fatty acid (22:5), fatty acid (23:0), Gb3(42:2), fatty acid (18:2), hydroxyl fatty acid (16:0), lysophosphatidylethanolamine (P-18:0), phosphatidylcholine (o-36:1), lysophosphatidylcholine (22:6), phosphatidylethanolamine (38:4), fatty acid (18:3), Gb3(34:1) and AcylCar (18:2); (12) fatty acid (22:6), fatty acid (22:5), fatty acid (23:0), Gb3(42:2), fatty acid (18:2), hydroxyl fatty acid (16:0), lysophosphatidylethanolamine (P-18:0), phosphatidylcholine (o-36:1), lysophosphatidylcholine (22:6), phosphatidylethanolamine (38:4), fatty acid (18:3), Gb3(34:1), AcylCar (18:2) and sphingomyelin (42:2); and (13) fatty acid (22:6), fatty acid (22:5), fatty acid (23:0), Gb3(42:2), fatty acid (18:2), hydroxyl fatty acid (16:0), lysophosphatidylethanolamine (P-18:0), phosphatidylcholine (o-36:1), lysophosphatidylcholine (22:6), phosphatidylethanolamine (38:4), fatty acid (18:3), Gb3(34:1), AcylCar (18:2), sphingomyelin (42:2) and GalCer(40:1)/GlcCer(40:1).

6. The cancer biomarker of claim 5, wherein the biomarker comprises fatty acid (22:6), fatty acid (22:5), fatty acid (23:0) and Gb3(42:2).

7. A method of determining whether a patient suffering from pleural effusion has cancer, the method comprising:
(i) measuring a concentration of a cancer biomarker for a patient suffering from pleural effusion, wherein the biomarker is at least two selected from the group consisting of: fatty acid (22:6), hydroxyl fatty acid (16:0), fatty acid (20:1), fatty acid (18:2), fatty acid (18:1), fatty acid (16:1), fatty acid (20:5), fatty acid (22:4), fatty acid (22:5), fatty acid (20:4) and fatty acid (20:2), and wherein the cancer is selected from lung cancer, breast cancer, gastric cancer and squamous cell carcinoma in a sample obtained from the patient;
(ii) comparing the concentration of the cancer biomarker in (i) with the concentration of the same cancer biomarker in a sample obtained from a control group,
wherein an increased concentration of the cancer biomarker in (i) as compared to the control group indicates that the patient has cancer,
wherein the control group comprises a patient suffering from pleural effusion without cancer, and
wherein the cancer is selected from lung cancer, breast cancer, gastric cancer and squamous cell carcinoma.

8. A method of treating cancer in a patient suffering from pleural effusion, the method comprising:
(i) measuring a concentration of a cancer biomarker for a patient suffering from pleural effusion, wherein the biomarker is at least two selected from the group consisting of: fatty acid (22:6), hydroxyl fatty acid (16:0), fatty acid (20:1), fatty acid (18:2), fatty acid (18:1), fatty acid (16:1), fatty acid (20:5), fatty acid (22:4), fatty acid (22:5), fatty acid (20:4) and fatty acid (20:2), and wherein the cancer is selected from lung cancer, breast cancer, gastric cancer and squamous cell carcinoma in a sample obtained from the patient;
(ii) comparing the concentration of the cancer biomarker in (i) with the concentration of the same cancer biomarker in a sample obtained from a control group, wherein the control group comprises a patient suffering from pleural effusion without cancer; and
(iii) administering to the patient at least one anti-cancer treatment, if there is an increased concentration of the cancer biomarker in (i) as compared to the control group;
wherein the cancer is selected from lung cancer, breast cancer, gastric cancer and squamous cell carcinoma.

9. A cancer biomarker for the detection of cancer with EGFR mutation, wherein the biomarker is at least two selected from the group consisting of: fatty acid (20:5), fatty acid (22:5), fatty acid (18:1), fatty acid (18:3), phosphatidylcholine (38:8), phosphatidylcholine (40:8), phosphatidylcholine (41:6), phosphatidylethanolamine (P-36:5), phosphatidylcholine (36:5), phosphatidylcholine (P-36:5), fatty acid (22:4), fatty acid (23:0), phosphatidylethanolamine (38:4), triacylglycerol 54:8 and Gb3(42:2), and wherein the cancer is selected from lung cancer, breast cancer, gastric cancer and squamous cell carcinoma.

10. The cancer biomarker of claim 7, wherein the biomarker is selected from the group consisting of: (1) phosphatidylcholine (41:6) and fatty acid (22:5); (2) phosphatidylcholine (41:6), fatty acid (22:5) and fatty acid (23:0); (3) phosphatidylcholine (41:6), fatty acid (22:5), fatty acid (23:0) and fatty acid (22:4); (4) phosphatidylcholine (41:6), fatty acid (22:5), fatty acid (23:0), fatty acid (22:4) and phosphatidylethanolamine (38:4); (5) phosphatidylcholine (41:6), fatty acid (22:5), fatty acid (23:0), fatty acid (22:4), phosphatidylethanolamine (38:4) and Gb3(42:2); (6) phosphatidylcholine (41:6), fatty acid (22:5), fatty acid (23:0), fatty acid (22:4), phosphatidylethanolamine (38:4), Gb3(42:2) and fatty acid (20:5); (7) phosphatidylcholine (41:6), fatty acid (22:5), fatty acid (23:0), fatty acid (22:4), phosphatidylethanolamine (38:4), Gb3(42:2), fatty acid (20:5) and fatty acid (18:1); (8) phosphatidylcholine (41:6), fatty acid (22:5), fatty acid (23:0), fatty acid (22:4), phosphatidylethanolamine (38:4), Gb3(42:2), fatty acid (20:5), fatty acid (18:1) and phosphatidylcholine (P-36:5); (9) phosphatidylcholine (41:6), fatty acid (22:5), fatty acid (23:0), fatty acid (22:4), phosphatidylethanolamine (38:4), Gb3(42:2), fatty acid (20:5), fatty acid (18:1), phosphatidylcholine (P-36:5) and fatty acid (18:3); (10) phosphatidylcholine (41:6), fatty acid (22:5), fatty acid (23:0), fatty acid (22:4), phosphatidylethanolamine (38:4), Gb3(42:2), fatty acid (20:5), fatty acid (18:1), phosphatidylcholine (P-36:5), fatty acid (18:3) and phosphatidylethanolamine (P-36:5); (11) phosphatidylcholine (41:6), fatty acid (22:5), fatty acid (23:0), fatty acid (22:4), phosphatidylethanolamine (38:4), Gb3(42:2), fatty acid (20:5), fatty acid (18:1), phosphatidylcholine (P-36:5), fatty acid (18:3), phosphatidylethanolamine (P-36:5) and phosphatidylcholine (38:8); (12) phosphatidylcholine (41:6), fatty acid (22:5), fatty acid (23:0), fatty acid (22:4), phosphatidylethanolamine (38:4), Gb3(42:2), fatty acid (20:5), fatty acid (18:1), phosphatidylcholine (P-36:5), fatty acid (18:3), phosphatidylethanolamine (P-36:5), phosphatidylcholine (38:8) and phosphatidylcholine (40:8); (13) phosphatidylcholine (41:6), fatty acid (22:5), fatty acid (23:0), fatty acid (22:4), phosphatidylethanolamine (38:4), Gb3(42:2), fatty acid (20:5), fatty acid (18:1), phosphatidylcholine (P-36:5), fatty acid (18:3), phosphatidylethanolamine (P-36:5), phosphatidylcholine (38:8), phosphatidylcholine (40:8) and triacylglycerol (54:8); and (14) phosphatidylcholine (41:6), fatty acid (22:5), fatty acid (23:0), fatty acid (22:4), phosphatidylethanolamine (38:4), Gb3(42:2), fatty acid (20:5), fatty acid (18:1), phosphatidylcholine (P-36:5), fatty acid (18:3), phosphatidylethanolamine (P-36:5), phosphatidylcholine (38:8), phosphatidylcholine (40:8), triacylglycerol (54:8) and phosphatidylcholine (36:5).

11. The cancer biomarker of claim 7, further comprising at least one selected from the group consisting of: fatty acid (20:3), fatty acid (20:4), fatty acid (22:6), lysophosphatidylethanolamine (P-16:0), phosphatidylethanolamine (P-38:5), fatty acid (16:2) and phosphatidylcholine (P-32:1).

12. The cancer biomarker of claim 10, wherein the biomarker comprises phosphatidylcholine (41:6), fatty acid (22:5), fatty acid (23:0), fatty acid (22:4), phosphatidylethanolamine (38:4), Gb3(42:2) and fatty acid (20:5).

13. A method of determining whether a patient suffering from cancer has EGFR mutation, the method comprising:
(i) measuring a concentration of a cancer biomarker for the detection of cancer with EGFR mutation, wherein the biomarker is at least two selected from the group consisting of: fatty acid (20:5), fatty acid (22:5), fatty acid (18:1), fatty acid (18:3), phosphatidylcholine (38:8), phosphatidylcholine (40:8), phosphatidylcholine (41:6), phosphatidylethanolamine (P-36:5), phosphatidylcholine (36:5), phosphatidylcholine (P-36:5), fatty acid (22:4), fatty acid (23:0), phosphatidylethanolamine (38:4), triacylglycerol 54:8 and Gb3(42:2), and wherein the cancer is selected from lung cancer, breast cancer, gastric cancer and squamous cell carcinoma in a sample obtained from the patient;
(ii) comparing the concentration of the cancer biomarker in (i) with the concentration of the same cancer biomarker in a sample obtained from a control group,
wherein an increased concentration of the cancer biomarker in (i) as compared to the control group indicates that the patient has EGFR mutation,
wherein the control group comprises a patient suffering from cancer without EGFR mutation, and
wherein the cancer is selected from lung cancer, breast cancer, gastric cancer and squamous cell carcinoma.

14. A method of treating cancer in a patient with EGFR mutation, the method comprising:
(i) measuring a concentration of a cancer biomarker for the detection of cancer with EGFR mutation, wherein the biomarker is at least two selected from the group consisting of: fatty acid (20:5), fatty acid (22:5), fatty acid (18:1), fatty acid (18:3), phosphatidylcholine (38:8), phosphatidylcholine (40:8), phosphatidylcholine (41:6), phosphatidylethanolamine (P-36:5), phosphatidylcholine (36:5), phosphatidylcholine (P-36:5), fatty acid (22:4), fatty acid (23:0), phosphatidylethanolamine (38:4), triacylglycerol 54:8 and Gb3(42:2), and wherein the cancer is selected from lung cancer, breast cancer, gastric cancer and squamous cell carcinoma in a sample obtained from the patient;
(ii) comparing the concentration of the cancer biomarker in (i) with the concentration of the same cancer biomarker in a sample obtained from a control group; and
(iii) administering to the patient at least one anti-cancer treatment for cancer with EGFR mutation if there is an increased concentration of the cancer biomarker in (i) as compared to the control group;
wherein the control group comprises a patient suffering from cancer without EGFR mutation, and
wherein the cancer is selected from lung cancer, breast cancer, gastric cancer and squamous cell carcinoma.

15. The biomarker of claim 1, wherein the cancer is lung cancer.

16. The biomarker of claim 15, wherein the lung cancer is non-small cell lung cancer.

17. The biomarker of claim 16, wherein the non-small cell lung cancer is non-small cell lung adenocarcinoma.

18. The method of claim 7, wherein the concentration of the biomarker is measured by targeted liquid chromatography mass spectrometry.

19. The method of claim 7, wherein the sample is a liquid sample.

20. The method of claim 19, wherein the liquid sample comprises pleural fluid.
